SEQUENCE LISTING (29 sequences)

SEQ ID NO: 1 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - DNA, 771 bp, Homo sapiens
atggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagc

SEQ ID NO: 3 - protein, 118 aa, Artificial Sequence
  Description of Artificial Sequence Synthetic polypeptide
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 4 - protein, 112 aa, Artificial Sequence
  Description of Artificial Sequence Synthetic polypeptide
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKR

SEQ ID NO: 5 - protein, 112 aa, Artificial Sequence
  Description of Artificial Sequence Synthetic polypeptide
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 6 - protein, 15 aa, Artificial Sequence
  Description of Artificial Sequence Synthetic peptide
KASQSVSFAGTSLMH

SEQ ID NO: 7 - protein, 7 aa, Artificial Sequence
  Description of Artificial Sequence Synthetic peptide
RASNLEA

SEQ ID NO: 8 - protein, 9 aa, Artificial Sequence
  Description of Artificial Sequence Synthetic peptide
QQSREYPYT

SEQ ID NO: 9 - protein, 5 aa, Artificial Sequence
  Description of Artificial Sequence Synthetic peptide
GYFMN

SEQ ID NO: 10 - protein, 17 aa, Artificial Sequence
  Description of Artificial Sequence Synthetic peptide
RIHPYDGDTFYNQKFQG

SEQ ID NO: 11 - protein, 10 aa, Artificial Sequence
  Description of Artificial Sequence Synthetic peptide
RIHPYDGDTF

SEQ ID NO: 12 - protein, 9 aa, Artificial Sequence
  Description of Artificial Sequence Synthetic peptide
YDGSRAMDY

SEQ ID NO: 13 - protein, 447 aa, Artificial Sequence
  Description of Artificial Sequence Synthetic polypeptide
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 218 aa, Artificial Sequence
  Description of Artificial Sequence Synthetic polypeptide
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 218 aa, Artificial Sequence
  Description of Artificial Sequence Synthetic polypeptide
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 17 aa, Artificial Sequence
  Description of Artificial Sequence Synthetic peptide
RIHPYDGDTFYNQNFKD

SEQ ID NO: 17 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 18 - protein, 20 aa, Homo sapiens
MQIPQAPWPVVWAVLQLGWR

SEQ ID NO: 19 - protein, 10 aa, Artificial Sequence
  Description of Artificial Sequence Synthetic peptide
GYTFTGYFMN

SEQ ID NO: 20 - protein, 5 aa, Artificial Sequence
  Description of Artificial Sequence Synthetic peptide
NYYMY

SEQ ID NO: 21 - protein, 17 aa, Artificial Sequence
  Description of Artificial Sequence Synthetic peptide
GINPSNGGTNFNEKFKN

SEQ ID NO: 22 - protein, 11 aa, Artificial Sequence
  Description of Artificial Sequence Synthetic peptide
RDYRFDMGFDY

SEQ ID NO: 23 - protein, 15 aa, Artificial Sequence
  Description of Artificial Sequence Synthetic peptide
RASKGVSTSGYSYLH

SEQ ID NO: 24 - protein, 7 aa, Artificial Sequence
  Description of Artificial Sequence Synthetic peptide
LASYLES

SEQ ID NO: 25 - protein, 9 aa, Artificial Sequence
  Description of Artificial Sequence Synthetic peptide
QHSRDLPLT

SEQ ID NO: 26 - protein, 120 aa, Artificial Sequence [sequence omitted]

SEQ ID NO: 27 - protein, 111 aa, Artificial Sequence [sequence omitted]

SEQ ID NO: 28 - protein, 447 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 218 aa, Artificial Sequence
  Description of Artificial Sequence Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2018/0333503 A1
(43) Pub. Date: Nov. 22, 2018

(54) ANTI-FOLR1 IMMUNOCONJUGATES AND ANTI-PD-1 ANTIBODY COMBINATIONS

(71) Applicants: ImmunoGen, Inc., Waltham, MA (US); Merck Sharp & Dohme Corp., Rahway, NJ (US)

(72) Inventor: Rodrigo R. RUIZ SOTO, Boston, MA (US)

(73) Assignees: ImmunoGen, Inc., Waltham, MA (US); Merck Sharp & Dohme Corp., Rahway, NJ (US)

(21) Appl. No.: 15/979,989

(22) Filed: May 15, 2018

Related U.S. Application Data
(60) Provisional application No. 62/506,940, filed on May 16, 2017.
(60) Provisional application No. 62/560,462, filed on Sep. 19, 2017.
(60) Provisional application No. 62/647,008, filed on Mar. 23, 2018.

(51) Int. Cl. A61K 47/68; A61P 35/04; A61K 9/00; A61K 39/395

(52) U.S. Cl. CPC A61K 47/6803; A61P 35/04; A61K 47/6869; A61K 31/57; A61K 9/0019; A61K 39/39558; A61K 2300/00; A61K 47/6889

(57) ABSTRACT

Therapeutic combinations of immunoconjugates that bind to FOLR1 (e.g., IMGN853) with anti-PD-1 antibodies or antigen-binding fragments thereof (e.g., pembrolizumab) are provided. Methods of administering the combinations to treat cancers, e.g., ovarian, peritoneal, or fallopian tube cancers, with greater clinical efficacy and/or decreased toxicity are also provided.

### FIELD OF THE INVENTION

The field of the invention generally relates to combinations of an anti-FOLR1 immunoconjugate with an anti-PD-1 antibody or antigen-binding fragment thereof (e.g., pembrolizumab) as well as the use of the combinations in the treatment of cancers, e.g., ovarian cancers.

### REFERENCE TO A SEQUENCE LISTING

A reference to a sequence listing which has been submitted electronically in ASCII format named 218110-0001-00-WO-574986_SL.txt, created on May 8, 2018, and is 29,561 bytes in size, is hereby incorporated by reference.

### BACKGROUND

Cancer is one of the leading causes of death in the developed world, with over one million people diagnosed with cancer and 500,000 deaths per year in the United States alone. Overall it is estimated that more than 1 in 3 people will develop some form of cancer during their lifetime.

Folate Receptor 1 (FOLR1), also known as Folate Receptor-alpha (FRα), or Folate Binding Protein, is a glycosylphosphatidylinositol (GPI)-anchored glycoprotein with a strong binding affinity for folic acid and reduced folic acid derivatives (see Leung et al., Clin. Biochem. 46:1462-1468 (2013)). FOLR1 mediates delivery of the physiological folate, 5-methyltetrahydrofolate, to the interior of cells. Expression of FOLR1 on normal tissues is restricted to the apical membrane of epithelial cells in the kidney proximal tubules, alveolar pneumocytes of the lung, bladder, testes, choroid plexus, and thyroid (Weitman S D, et al., Cancer Res. 52:3396-3401 (1992); Antony A C, Ann. Rev. Nutr. 16:501-521 (1996); Kalli K R, et al., Gynecol. Oncol. 108:619-626 (2008)). FOLR1 is overexpressed in epithelial-derived tumors including ovarian, uterine, breast, endometrial, pancreatic, renal, lung, colorectal, and brain tumors. This expression pattern of FOLR1 makes it a desirable target for FOLR1-directed cancer therapy.

Programmed death receptor 1 (PD-1) is an immunoinhibitory receptor that is primarily expressed on activated T and B cells. Interaction with its ligands has been shown to attenuate T-cell responses. Blockade of the interaction between PD-1 and one of its ligands, PD-L1, has been shown to enhance tumor-specific CD8+ T-cell immunity and may therefore be helpful in clearance of tumor cells by the immune system. PD-1 has been shown to negatively regulate antigen receptor signaling upon engagement of its ligands (PD-L1 and/or PD-L2). In addition, studies have shown that interaction of PD-1 with its ligands leads to the inhibition of lymphocyte proliferation. Disruption of the PD-1/PD-L1 interaction has been shown to increase T cell proliferation and cytokine production and block progression of the cell cycle. Thus, it was hypothesized that therapeutic blockade of the PD-1 pathway may be helpful in overcoming immune tolerance and that such a selective blockade may be of use in the treatment of cancer.

In human studies, R. M. Wong et al. (Int. Immunol. 19: 1223-1234 (2007)) showed that PD-1 blockade using a fully human anti-PD-1 antibody augmented the absolute numbers of tumor-specific CD8+ T cells (CTLs) in ex vivo stimulation assays using vaccine antigens and cells from vaccinated individuals. In a similar study, antibody blockade of PD-L1 resulted in enhanced cytolytic activity of tumor-associated antigen-specific cytotoxic T cells and increased cytokine production by tumor specific T_{H} cells (Blank C. et al., Int. J. Cancer 119: 317-327 (2006)). In 2014, the anti-PD-1 antibody pembrolizumab (Keytruda®) was approved by the United States Food and Drug Agency (US FDA) for the treatment of patients with unresectable or metastatic melanoma. Pembrolizumab was then subsequently approved for the treatment of certain patients with metastatic non-small cell lung cancer (NSCLC), recurrent or metastatic head and neck squamous cell cancer and refractory classical Hodgkin lymphoma.

Despite the recent developments, the prognosis for many patients with cancers, and in particular ovarian cancer, fallopian tube cancer, and cancer of the peritoneum, remains poor, there is a clear unmet medical need for more effective therapies that can, for example, achieve a high objective response rate as well as durable responses.

### BRIEF SUMMARY OF THE INVENTION

The present invention relates to the discovery that a combination of 6 mg/kg AIBW of IMGN853 (mirvetuximab soravtansine) and 200 mg pembrolizumab (Keytruda®) is effective for the treatment of ovarian cancer, fallopian tube cancer, and cancer of the peritoneum. Accordingly, combinations of an anti-FOLR1 immunoconjugate (e.g. IMGN853) with an anti-PD-1 antibody or antigen-binding fragment thereof (e.g., pembrolizumab) are provided herein. To date, there is no clinical data available regarding treatment using combinations of an anti-FOLR1 immunoconjugate with a checkpoint inhibitor such as an anti-PD-1 antibody or antigen-binding fragment thereof.

Methods for treating a patient having ovarian cancer, peritoneal cancer, endometrial cancer or fallopian tube cancer are provided herein. In some embodiments, the method comprises administering to the patient in need thereof an immunoconjugate that binds to FOLR1, wherein the immunoconjugate comprises a maytansinoid and an anti-FOLR1 antibody or antigen-binding fragment thereof comprising a heavy chain variable region (VH) complementary determining region (CDR)1 sequence of SEQ ID NO:9, a VH CDR2 sequence of SEQ ID NO:10, and a VH CDR3 sequence of SEQ ID NO: 12, and a light chain variable region (VL) CDR1 sequence of SEQ ID NO: 6, a VL CDR2 sequence of SEQ ID NO: 7, and a VL CDR3 sequence of SEQ ID NO: 8, and an anti-PD-1 antibody or antigen-binding fragment thereof comprising a VH CDR1 sequence of SEQ ID NO: 20, a VH CDR2 sequence of SEQ ID NO: 21, and a VH CDR3 sequence of SEQ ID NO: 22, and a VL CDR1 sequence of SEQ ID NO: 23, a VL CDR2 sequence of SEQ ID NO: 24, and a VL CDR3 sequence of SEQ ID NO: 25.

In some embodiments, the anti-FOLR1 antibody or antigen-binding fragment thereof comprises a VH comprising the sequence of SEQ ID NO: 3 and a VL comprising the sequence of SEQ ID NO: 5. In some embodiements, the anti-FOLR1 antibody or antigen-binding fragment comprises a heavy chain comprising the sequence of SEQ ID NO: 13 and a light chain comprising the sequence SEQ ID NO: 15. In some embodiments, the maytansinoid is DM4. In some embodiments, the maytanisonid is linked to the antibody or antigen-binding fragement thereof by a sulfo-SPDB linker.

In some embodiments, the method for treating a patient having ovarian cancer, peritoneal cancer, or fallopian tube cancer comprising administering to the patient in need thereof an immunoconjugate that binds to FOLR1, wherein the immunoconjugate comprises a maytansinoid and an anti-FOLR1 antibody or antigen-binding fragment thereof comprising (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774; and an anti-PD-1 antibody or antigen-binding fragment thereof comprising a VH CDR1 sequence of SEQ ID NO:20, a VH CDR2 sequence of SEQ ID NO:21, and a VH CDR3 sequence of SEQ ID NO:22, and a VL CDR1 sequence of SEQ ID NO:23, a VL CDR2 sequence of SEQ ID NO:24, and a VL CDR3 sequence of SEQ ID NO:25. In some embodiments, the the maytansinoid is DM4, and wherein the DM4 is linked to the antibody by sulfo-SPDB. In some embodiments, the immunoconjugate comprises 1-10 maytansinoid molecules, 2-5 maytansinoid molecules, or 3-4 maytansinoid molecules. In some embodiments, the maytansinoid (e.g., DM4) is linked to the anti-FOLR1 antibody or antigen-binding fragment thereof via a lysine residue of the antibody or antigen-binding fragment thereof. In some embodiments, the 1-10, 2-5, or 3-4 maytansinoid molecules (e.g., DM4) are attached to the anti-FOLR1 antibody or antigen-binding fragment thereof via lysine residues of the antibody or antigen-binding fragment thereof.

In some embodiments, the immunoconjugate has the following chemical structure:

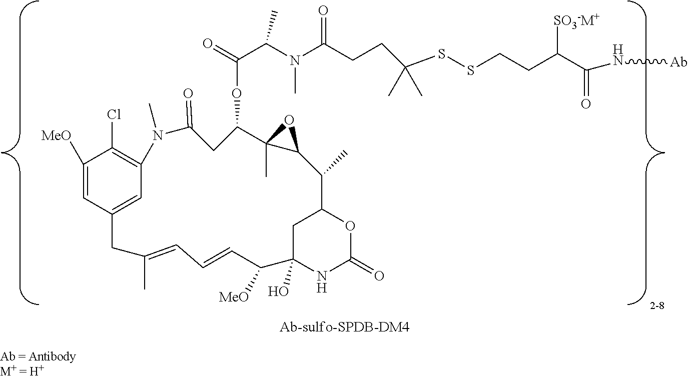

wherein “Ab” represents the anti-FOLR1 antibody or antigen binding fragment thereof.

In some embodiments, the 2-8 maytansinoids (e.g., DM4) are linked to the anti-FOLR1 antibody or antigen-binding fragment thereof via a lysine residue of the antibody or antigen-binding fragment thereof.

In some embodiments, the immunoconjugate comprises 2-5 or 3-4 maytansinoid molecules. In some embodiments, the 2-5 or 3-4 maytansinoid molecules (e.g., DM4) are attached to the anti-FOLR1 antibody or antigen-binding fragment thereof via lysine residues of the antibody or antigen-binding fragment thereof. In some embodiments, the immunoconjugate is administered once every three weeks. In some embodiments, the immunoconjugate is administered at a dose of about 6 mg/kg AIBW. In some embodiments, the immunoconjugate is administered at a dose of about 5 mg/kg AIBW. In some embodiments, the anti-PD-1 antibody or antigen-binding fragment thereof comprises a VH comprising the sequence of SEQ ID NO:26 and a VL comprising the sequence of SEQ ID NO:27. In some embodiments, the anti-PD-1 antibody or antigen-binding fragment thereof is pembrolizumab. In some embodiments, the anti-PD-1 antibody or antigen-binding fragment thereof is administered once every 3 weeks. In some embodiments, the anti-PD-1 antibody or antigen-binding fragment thereof is administered at a dose of about 200 mg.

In some embodiments, the cancer is ovarian cancer. In some embodiments, the ovarian cancer is epithelial ovarian cancer. In some embodiments, the ovarian cancer is platinum resistant, relapsed, or refractory. In some embodiments, the administration results in a decrease in CA125.

In some embodiments, the cancer is peritoneal cancer. In some embodiments, the peritoneal cancer is primary peritoneal cancer.

In some embodiments, the cancer is endometrial cancer. In some embodiments, the endometrial cancer is serous endometrial cancer. In some embodiments, the endometrial cancer is endometriod endometrial cancer.

In some embodiments, expression of FRα is low, medium, or high. Low expression refers to a range of at least 25% of cells to 49% of cells in the sample obtained from the patient having an IHC score of 2 or 3. Medium expression refers to a range of at least 50% of cells to 74% of cells in the sample obtained from the patient having an IHC score of 2 or 3. High expression refers to a range of 75% or more cells in the sample obtained from the patient having an IHC score of 2 or 3. The method of treatment described herein may be wherein the patient sample has an IHC score of at least 2, and wherein at least 25% to no more than 49% of the cells in the patient sample has an IHC score of at least 2. The method of treatment may be wherein the patient sample has an IHC score of at least 2, and wherein at least 50% to no more than 74% of the cells in the patient sample has an IHC score of at least 2. The method of treatment may be wherein the patient sample has an IHC score of at least 2, and wherein at least 75% to 100% of the cells in the patient sample has an IHC score of at least 2.

According to an immunohistochremistry visual scoring system, a patient may be determined to be FRα positive. FRα positive may refer to greater than or equal to 50% of tumor cells with FOLR1 membrane staining visible at less than or equal to 10× microscope objective. The methods of treatment described herein may be for a patient described as having medium or high FRα expression.

A patient may be determined to be FOLR1 positive and be referred to as having FOLR1 positive status.

In some embodiments, the cancer expresses PD-L1.

In some embodiments, the patient has at lease one lesion that meets the definition of measurable disease according to RECIST 1.1.

In some embodiments, the immunoconjugate and the anti-PD-1 antibody or antigen-binding fragment thereof are administered sequentially in separate pharmaceutical compositions. In some embodiments, the immunoconjugate is administered before the anti-PD-1 antibody or antigen-binding fragment thereof.

In some embodiments, the immunoconjugate is administered intravenously or intraperitoneally. In some embodiments, the anti-PD-1 antibody or antigen-binding fragment thereof is administered intravenously. In some embodiments, administration is a first-line therapy. In some embodiments, administration is a second-line therapy. In some embodiments, the administration is a third-line or later than third line therapy. In some embodiments, the patient has previously been treated with a platinum compound, a taxane, bevacizumab, a PARP inhibitor, or a combination thereof. In some embodiments, the cancer is primary platinum refractory. In some embodiments, the cancer is platinum resistant. In some embodiments, the cancer is platinum sensitive. In some embodiments, the cancer is metastatic or advanced.

In some embodiments, administration of the immunoconjugate with the anti-PD-1 antibody or antigen-binding fragment thereof produces a greater therapeutic benefit than administration of the immunoconjugate alone or the anti-PD-1 antibody or antigen-binding fragment thereof alone. In some embodiments, administration of the immunoconjugate and the anti-PD-1 antibody or antigen-binding fragment thereof does not produce more toxicity than administration of the immunoconjugate alone or the anti-PD-1 antibody or antigen-binding fragment thereof alone.

In some embodiments, the method further comprises administering a steroid to the patient. In some embodiments, the steroid is administered prior to the administration of the immunoconjugate. In some embodiments, the steroid is administered about 30 minutes prior to the administration of the immunoconjugate. In some embodiments, the steroid is a corticosteroid. In some embodiments, the steroid is dexamethasone. In some embodiments, the steroid is administered orally, intravenously, or a combination thereof. In some embodiments, the steroid is administered as an eye drop. In some embodiements, the eye drop is a lubricating eyedrop. In some embodiments, the method further comprises administering acetaminophen, diphenhydramine, or a combination thereof to the patient.

In some embodiments, the methods comprises treating a patient having ovarian, peritoneal, or fallopian tube cancer comprising administering to the patient in need thereof 6 mg/AIBW kg of an immunoconjugate that binds to FOLR1 and 200 mg of pembrolizumab, wherein the immunoconjugate that binds to FOLR1 comprises an antibody linked to the maytansinoid DM4 by a sulfo-SPDB linker, wherein the an antibody comprises (i) a heavy chain comprising the sequence of SEQ ID NO:13 and (ii) a light chain comprising the sequence of SEQ ID NO:15.

In some embodiments, the methods comprises treating a patient having ovarian, peritoneal, or fallopian tube cancer comprising administering to the patient in need thereof 5 mg/AIBW kg of an immunoconjugate that binds to FOLR1 and 200 mg of pembrolizumab, wherein the immunoconjugate that binds to FOLR1 comprises an antibody linked to the maytansinoid DM4 by a sulfo-SPDB linker, wherein the an antibody comprises (i) a heavy chain comprising the sequence of SEQ ID NO:13 and (ii) a light chain comprising the sequence of SEQ ID NO:15.

In some embodiments, the method comprises treating a patient having ovarian, peritoneal, or fallopian tube cancer comprising administering to the patient in need thereof 6 mg/AIBW kg of an immunoconjugate that binds to FOLR1 and 200 mg of pembrolizumab, wherein the immunoconjugate that binds to FOLR1 comprises an antibody linked to the maytansinoid DM4 by a sulfo-SPDB linker, wherein the an antibody comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774.

In some embodiments, the method comprises treating a patient having ovarian, peritoneal, or fallopian tube cancer comprising administering to the patient in need thereof 5 mg/AIBW kg of an immunoconjugate that binds to FOLR1 and 200 mg of pembrolizumab, wherein the immunoconjugate that binds to FOLR1 comprises an antibody linked to the maytansinoid DM4 by a sulfo-SPDB linker, wherein the an antibody comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774.

In some embodiments, the immunoconjugate comprises 1-10, 2-5, or 3-4 maytansinoids. In some embodiments, the immunoconjugate has the following chemical structure:

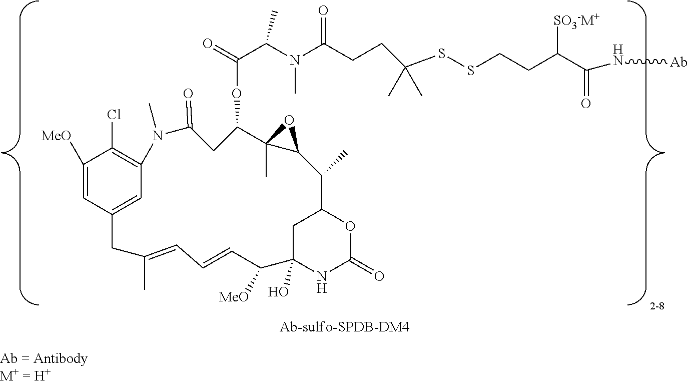

wherein “Ab” represents the anti-FOLR1 antibody or antigen binding fragment thereof.

In some embodiments, the 2-8 maytansinoid molecules (e.g., DM4) are attached to the antibody via lysine residues of the antibody. In some embodiements, the immunoconjugate comprises 2-5 or 3-4 maytansinoids. In some embodiements, at least 25% of cells in a tumor sample obtained from the patient have an FOLR1 IHC score of at least 2. In some embodiements, the immunoconjugate and the pembrolizumab are administered intravenously, and the immunoconjugate is administered before the pembrolizumab. In some embodiements, a steroid is administered prior to the administration of the immunoconjugate.

### DESCRIPTION OF THE FIGURES

FIGS. 1A, 1B, and 1C depict the percent tumor change in target lesions in a patient by FRα expression level. FIG. 1A depicts low FRα. FIG. 1B depicts medium FRα. FIG. 1C depicts high FRα.

### DETAILED DESCRIPTION OF THE INVENTION

The present invention provides combinations of an anti-FOLR1 immunoconjugate with an anti-PD-1 antibody or antigen-binding fragment thereof (e.g., pembrolizumab) and the use of the combinations in the treatment of cancer.

### I. Definitions

To facilitate an understanding of the present invention, a number of terms and phrases are defined below.

The term “FOLR1” as used herein refers to any native human FOLR1 polypeptide, unless otherwise indicated. FOLR1 is also referred to as “human folate receptor 1,” “folate receptor alpha (FR-α),” and “FRα”. The term “FOLR1” encompasses “full-length,” unprocessed FOLR1 polypeptide as well as any form of FOLR1 polypeptide that results from processing within the cell. The term also encompasses naturally occurring variants of FOLR1, e.g., those encoded by splice variants and allelic variants. The FOLR1 polypeptides described herein can be isolated from a variety of sources, such as from human tissue types or from another source, or prepared by recombinant or synthetic methods. Where specifically indicated, “FOLR1” can be used to refer to a nucleic acid that encodes a FOLR1 polypeptide. Human FOLR1 sequences are known and include, for example, the sequences publically available at UniProtKB Accession No. P15328 (including isoforms). As used herein, the term “human FOLR1” refers to FOLR1 comprising the sequence of SEQ ID NO:1.

The term “PD-1” as used herein, refers to any native human PD-1 polypeptide, unless otherwise indicated. PD-1 is also referred to Programmed death protein 1 or Programmed cell death protein 1. The term “PD-1” encompasses “full-length,” unprocessed PD-1 polypeptide as well as any form of PD-1 polypeptide that results from processing in the cell. The term also encompasses naturally occurring variants of PD-1, e.g., those encoded by splice variants and allelic variants. The PD-1 polypeptides described herein can be isolated from a variety of sources, such as from human tissue types or from another source, or prepared by recombinant or synthetic methods. Where specifically indicated, “PD-1” can be used to refer to a nucleic acid that encodes a PD-1 polypeptide. Human PD-1 sequences are known and include, for example, the sequences publically available at UniProtKB Accession No. P15692. As used herein, the term “human PD-1” refers to PD-1 comprising the sequence of SEQ ID NO:17 or a variant thereof.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

A “blocking” antibody or an “antagonist” antibody is one which inhibits or reduces biological activity of the antigen it binds, such as FOLR1 or PD-1. In some embodiments, blocking antibodies or antagonist antibodies substantially or completely inhibit the biological activity of the antigen. The biological activity can be reduced by 10%, 20%, 30%, 50%, 70%, 80%, 90%, 95%, or even 100%.

The term “anti-FOLR1 antibody” or “an antibody that binds to FOLR1” refers to an antibody that is capable of binding FOLR1 with sufficient affinity such that the antibody is useful as a diagnostic and/or therapeutic agent in targeting FOLR1 (e.g., the huMov19 (M9346A) antibody). The extent of binding of an anti-FOLR1 antibody to an unrelated, non-FOLR1 protein can be less than about 10% of the binding of the antibody to FOLR1 as measured, e.g., by a radioimmunoassay (RIA).

The term “anti-PD-1 antibody” or “an antibody that binds to PD-1” refers to an antibody that is capable of specifically binding PD-1 with sufficient affinity such that the antibody is useful as a therapeutic agent in targeting PD-1 (e.g., pembrolizumab). The extent of binding of an anti-PD-1 antibody to an unrelated, non-PD-1 protein can be less than about 10% of the binding of the antibody to PD-1 as measured, e.g., by a radioimmunoassay (RIA). In certain embodiments, an antibody that binds to PD-1 has a dissociation constant (Kd) of ≤1 μM, ≤100 nM, ≤10 nM, ≤1 nM, or ≤0.1 nM. In certain embodiments, the antibody or antigen-binding fragment thereof that binds to PD-1 is pembrolizumab. In certain embodiments, the antibody or antigen-binding fragment thereof that binds to PD-1 is highly similar to pembrolizumab and has no clinically meaningful differences with respect to safety and effectiveness as compared to pembrolizumab.

The term “pembrolizumab” refers to a specific anti-PD-1 antibody. Pembrolizumab is a recombinant humanized monoclonal IgG₄-κ isotype antibody that blocks the interaction between PD-1 and its ligands PD-L1 and PD-L2 (see Sul J., et al., The Oncologist 21: 1-8 (2016), U.S. Pat. No. 8,354,509 and U.S. Pat. No. 8,900,587). Pembrolizumab is the active ingredient in Keytruda® (Merck &Co., Inc. Whitehouse Station, N.J., USA). Pembrolizumab is an anti-PD1 antibody that contains the three light chain CDR-1, -2, and -3 sequences of SEQ ID NO:23, SEQ ID NO:24, and SEQ ID NO:25, respectively, and the three heavy chain CDR-1, -2, and -3 sequences of SEQ ID NO:20, SEQ ID NO:21, and SEQ ID NO:22, respectively. Pembrolizumab also contains the variable light chain sequence of SEQ ID NO:27 and the variable heavy chain sequence of SEQ ID NO:26.

The terms “line of treatment” or “line of therapy” refer to a therapeutic regimen that can include but is not limited to surgery, radiation therapy, chemotherapy, differentiating therapy, biotherapy, immune therapy, or the administration of one or more anti-cancer agents (e.g., a cytotoxic agent, an anti-proliferative compound, and/or an angiogenesis inhibitor).

The terms “first-line treatment,” “first-line therapy,” and “front-line therapy” refer to the preferred and standard initial treatment for a particular condition, e.g., a given type and stage of cancer. These treatments differ from “second-line” therapies, which are tried when a first-line therapy does not work adequately. “Third-line” therapies are tried when a first-line therapy and a second-line therapy do not work adequately.

For example, the combination of an anti-FOLR1 immunoconjugate (e.g., IMGN853) with an anti-PD-1 antibody or antigen-binding fragment thereof (e.g., pembrolizumab) provided herein can be given as a first-line therapy, a second-line therapy (e.g., in patients with platinum sensitive or platinum resistant epithelial ovarian, fallopian tube, or peritoneal cancer), or a third-line therapy (e.g., in patients with platinum sensitive or platinum resistant epithelial ovarian, fallopian tube, or peritoneal cancer). The combination of a FOLR1 immunoconjugate (e.g., IMGN853) with an anti-PD-1 antibody or antigen-binding fragment thereof (e.g., pembrolizumab) provided herein can be given as a line of therapy in patients having received 0, 1, 2, 3, 4, 5, 6, or more lines of therapy prior to treatment with the combination of a FOLR1 immunoconjugate (e.g., IMGN853) and an anti-PD-1 antibody or antigen-binding fragment thereof (e.g., pembrolizumab) provided herein. The combination of a FOLR1 immunoconjugate (e.g., IMGN853) with an anti-PD-1 antibody or antigen-binding fragment thereof (e.g., pembrolizumab) provided herein can be given as a line of therapy in patients having received at least 1, at least 2, or at least 3 lines of therapy prior to treatment with the combination of a FOLR1 immunoconjugate (e.g., IMGN853) and an anti-PD-1 antibody or antigen-binding fragment thereof (e.g., pembrolizumab) provided herein. In some embodiments, the combination of a FOLR1 immunoconjugate (e.g., IMGN853) and an anti-PD-1 antibody or antigen-binding fragment thereof (e.g., pembrolizumab) provided herein can be given as a line of therapy in patients having received no more than 1, no more than 2, no more than 3, no more than 4, no more than 5, or no more than 6 lines of therapy. In certain embodiments, the combination of a FOLR1 immunoconjugate (e.g., IMGN853) and an anti-PD-1 antibody or antigen-binding fragment thereof (e.g., pembrolizumab) provided herein can be given as an adjuvant therapy a neoadjuvant therapy, or a maintenance therapy.

The term “adjuvant therapy” refers to systemic therapy given after surgery. Adjuvant therapy, in the broadest sense, is treatment given in addition to the primary therapy to kill any cancer cells that may have spread, even if the spread cannot be detected by radiologic or laboratory tests.

The term “neoadjuvant therapy” refers to systemic therapy given prior to surgery.

The term “maintenance therapy” refers to therapy that is given to help keep cancer from coming back after it has disappeared following the initial therapy.

The term “IMGN853” (also known as mirvetuximab soravtansine) refers to the immunoconjugate described herein containing the huMov19 (M9346A) antibody, the sulfoSPDB linker, and the DM4 maytansinoid. The huMov19 (M9346A) antibody is an anti-FOLR1 antibody comprising the variable heavy chain sequence SEQ ID NO:3 and the variable light chain sequence SEQ ID NO:5. DM4 refers to N2′-deacetyl-N2′-(4-mercapto-4-methyl-1-oxopentyl) maytansine. “SulfoSPDB” refers to the N-succinimidyl 4-(2-pyridyldithio)-2-sulfobutanoate) linker.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “humanized” antibody or antigen-binding fragment thereof refers to forms of non-human (e.g., murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g., mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3): 969-973 (1994), and Roguska et al., Protein Eng. 9(10): 895-904 (1996). In some embodiments, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.), “Kabat”); and (2) an approach based on crystallographic studies of antigen-antibody complexes (Al-lazikani et al, J. Molec. Biol. 273: 927-948 (1997)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest. (5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. (5th Ed., 1991, National Institutes of Health, Bethesda, Md.), “Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196: 901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human” antibody or antigen-binding fragment thereof means an antibody or antigen-binding fragment thereof produced by a human or an antibody or antigen-binding fragment thereof having an amino acid sequence corresponding to an antibody or antigen-binding fragment thereof produced by a human made using any technique known in the art. This definition of a human antibody or antigen-binding fragment thereof includes intact or full-length antibodies and fragments thereof.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g., mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better”, the antibody's affinity for the antigen is <0.6 nM, i.e. 0.59 nM, 0.58 nM, 0.57 nM etc. or any value less than 0.6 nM.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

The phrase “substantially similar,” or “substantially the same”, as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristic measured by said values (e.g., Kd values). The difference between said two values can be less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “immunoconjugate” or “conjugate” as used herein refers to a compound or a derivative thereof that is linked to a cell binding agent (i.e., an anti-FOLR1 antibody or fragment thereof) and is defined by a generic formula: C-L-A, wherein C=cytotoxin, L=linker, and A=antibody or antigen-binding fragment thereof e.g., an anti-FOLR1 antibody or antibody fragment. Immunoconjugates can also be defined by the generic formula in reverse order: A-L-C.

A “linker” is any chemical moiety that is capable of linking a compound, usually a drug (such as a maytansinoid), to a cell-binding agent (such as an anti FOLR1 antibody or a fragment thereof) in a stable, covalent manner. Linkers can be susceptible to or be substantially resistant to, e.g., disulfide bond cleavage, at conditions under which the compound or the antibody remains active. Suitable linkers are well known in the art and include, for example, disulfide groups and thioether groups.

The terms “cancer” and “cancerous” refer to or describe the physiological condition in mammals in which a population of cells are characterized by unregulated cell growth. Examples of cancer include ovarian cancer, fallopian tube cancer, and peritoneal cancer. Another example of cancer is endometrial cancer. The cancer can be a cancer that expresses FOLR1 (“FOLR1-expressing cancer” or “FRα positive” cancer).

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

An “advanced” cancer is one which has spread outside the site or organ of origin, either by local invasion or metastasis. The term “advanced” cancer includes both locally advanced and metastatic disease.

“Metastatic” cancer refers to cancer that has spread from one part of the body to another part of the body.

A “refractory” cancer is one that progresses even though an anti-tumor treatment, such as a chemotherapy, is administered to the cancer patient. An example of a refractory cancer is one which is platinum refractory.

A patient is “platinum-refractory” if the patient does not respond to platinum-based therapy and shows progression during the course of therapy or within 4 weeks after the last dose. “Platinum-resistant” patients progress within 6 months of platinum-based therapy. “Partially platinum-sensitive” patients progress between 6 and 12 months of platinum-based therapy. “Platinum-sensitive” patients progress within an interval of 12 months or more.

A “recurrent” cancer is one that has regrown, either at the initial site or at a distant site, after a response to initial therapy.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

A “relapsed” patient is one who has signs or symptoms of cancer after remission. Optionally, the patient has relapsed after adjuvant or neoadjuvant therapy.

Administration “in combination with” one or more further therapeutic agents includes simultaneous (concurrent) or consecutive administration in any order.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered serially, by alternation, or in parallel as separate formulations; or (3) by some other regimen. When delivered in alternation therapy, a synergistic effect can be attained when the compounds are administered or delivered sequentially, e.g., by different injections in separate syringes. A synergistic combination produces effects that are greater than the additive effects of the individual components of the combination.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

An “effective amount” of an antibody, immunoconjugate, or other drug as disclosed herein is an amount sufficient to carry out a specifically stated purpose. An “effective amount” can be determined empirically and in a routine manner, in relation to the stated purpose.

The term “therapeutically effective amount” refers to an amount of an antibody, immunoconjugate, or other drug effective to “treat” a disease or disorder in a subject or mammal. In the case of cancer, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), a decrease in CA125 in the case of ovarian cancer or any combination thereof. See the definition herein of “treating”. To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “respond favorably” generally refers to causing a beneficial state in a subject. With respect to cancer treatment, the term refers to providing a therapeutic effect on the subject. Positive therapeutic effects in cancer can be measured in a number of ways (See, W. A. Weber, J. Nucl. Med. 50:1S-10S (2009)). For example, tumor growth inhibition, molecular marker expression, serum marker expression, and molecular imaging techniques can all be used to assess therapeutic efficacy of an anti-cancer therapeutic. Log₁₀ Cell Kill (LCK) can be used to quantify the tumor cell kill. Log₁₀ cell kill (LCK) is calculated with the formula LCK=(T−C)/T_{d}×3.32, where (T−C) (or tumor growth delay (TGD)) is the median time (in days) for the treatment group and control group tumors to reach a predetermined size (tumor-free survivors excluded). T_{d} is the tumor doubling time (estimated from nonlinear exponential curve fit of daily median of control tumor growth), and 3.32 is the number of cell doublings per log of cell growth. The ability to reduce tumor volume may be assessed, for example, by measuring a % T/C value, which is the median tumor volume of treated subjects divided by the median tumor volume of the control subjects. With respect to tumor growth inhibition, according to NCI standards, a T/C≤42% is the minimum level of anti-tumor activity. A T/C<10% is considered a high anti-tumor activity level, with T/C(%)=Median tumor volume of the treated/Median tumor volume of the control×100. A favorable response can be assessed, for example, by increased progression-free survival (PFS), disease-free survival (DFS), or overall survival (OS), complete response (CR), partial response (PR), or, in some cases, stable disease (SD), a decrease in progressive disease (PD), a reduced time to progression (TTP), a decrease in CA125 in the case of ovarian cancer, or any combination thereof.

PFS, DFS, and OS can be measured by standards set by the National Cancer Institute and the U.S. Food and Drug Administration for the approval of new drugs. See Johnson et al, J. Clin. Oncol. 21(7): 1404-1411 (2003).

“Progression free survival” (PFS) refers to the time from enrollment to disease progression or death. PFS is generally measured using the Kaplan-Meier method and Response Evaluation Criteria in Solid Tumors (RECIST) 1.1 standards. Generally, progression free survival refers to the situation wherein a patient remains alive, without the cancer getting worse.

“Time to Tumor Progression” (TTP) is defined as the time from enrollment to disease progression. TTP is generally measured using the RECIST 1.1 criteria.

A “complete response” or “complete remission” or “CR” indicates the disappearance of all signs of tumor or cancer in response to treatment. This does not always mean the cancer has been cured.

A “partial response” or “PR” refers to a decrease in the size or volume of one or more tumors or lesions, or in the extent of cancer in the body, in response to treatment.

“Stable disease” refers to disease without progression or relapse. In stable disease there is neither sufficient tumor shrinkage to qualify for partial response nor sufficient tumor increase to qualify as progressive disease.

“Progressive disease” refers to the appearance of one more new lesions or tumors and/or the unequivocal progression of existing non-target lesions. Progressive disease can also refer to a tumor growth of more than 20% since treatment began, either due to an increases in mass or in spread of the tumor.

“Disease free survival” (DFS) refers to the length of time during and after treatment that the patient remains free of disease.

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of diagnosis or treatment. In a population of patients, overall survival is measaured as mean overall survival (mOS).

By “extending survival” or “increasing the likelihood of survival” is meant increasing PFS and/or OS in a treated subject relative to an untreated subject or relative to a control treatment protocol, such as those used in the standard of care for a type of cancer.

A “decrease in CA125 levels” can be assessed according to the Gynecologic Cancer Intergroup (GCIG) guidelines. For example, CA125 levels can be measured prior to treatment to establish a baseline CA125 level. CA125 levels can be measured one or more times during or after treatment, and a reduction in the CA125 levels over time as compared to the baseline level is considered a decrease in CA125 levels.

The term “increased expression” or “overexpression” of FOLR1 in a particular tumor, tissue, or cell sample refers to FOLR1 (a FOLR1 polypeptide or a nucleic acid encoding such a polypeptide) that is present at a level higher than that which is present in a healthy or non-diseased (native, wild type) tissue or cells of the same type or origin. Such increased expression or overexpression can be caused, for example, by mutation, gene amplification, increased transcription, increased translation, or increased protein stability.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In certain embodiments, a subject is successfully “treated” for cancer according to the methods of the present invention if the patient shows one or more of the following: a reduction in the number of or complete absence of cancer cells; a reduction in the tumor burden; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), a decrease in CA125 in the case of ovarian cancer, or any combination thereof.

Prophylactic or preventative measures refer to measures that prevent and/or slow the development of a targeted pathological condition or disorder. Thus, those in need of prophylactic or preventative measures include those prone to have the disorder and those in whom the disorder is to be prevented.

The term “instructing” means providing directions for applicable therapy, medication, treatment, treatment regimens, and the like, by any means, for example, in writing, such as in the form of package inserts or other written promotional material.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies of the invention do not abrogate the binding of the polypeptide or antibody containing the amino acid sequence, to the antigen(s), i.e., the FOLR1 or VEGF to which the polypeptide or antibody binds. Methods of identifying nucleotide and amino acid conservative substitutions which do not eliminate antigen binding are well-known in the art (see, e.g., Brummell et al., Biochem. 32: 1180-1 187 (1993); Kobayashi et al., Protein Eng. 12(10): 879-884 (1999); and Burks et al., Proc. Natl. Acad. Sci. USA 94:.412-417 (1997)).

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Anti-FOLR1 Immunoconjugates

Described herein are methods of administering immunoconjugates that specifically bind FOLR1 (e.g., IMGN853). These agents are referred to herein as “FOLR1-immunoconjugates or anti-FOLR1 immunoconjugates.” The amino acid and nucleotide sequences for human FOLR1 are known in the art and are also provided herein as SEQ ID NO:1 and SEQ ID NO:2, respectively.

SEQ ID NO: 1
human folate receptor 1
MAQRMTTQLLLLLVWVAVVGEAQTRIAWARTELLNVCMNAKHHKEKPGPE
DKLHEQCRPWRKNACCSTNTSQEAHKDVSYLYRENWNHCGEMAPACKRHF
IQDTCLYECSPNLGPWIQQVDQSWRKERVLNVPLCKEDCEQWWEDCRTSY
TCKSNWHKGWNWTSGFNKCAVGAACQPFHFYFPTPTVLCNEIWTHSYKVS
NYSRGSGRCIQMWFDPAQGNPNEEVARFYAAAMSGAGPWAAWPFLLSLAL
MLLWLLS
SEQ ID NO: 2
human folate receptor 1 nucleic acid sequence
atggctcagcggatgacaacacagctgctgctccttctagtgtgggtggc
tgtagtaggggaggctcagacaaggattgcatgggccaggactgagcttc
tcaatgtctgcatgaacgccaagcaccacaaggaaaagccaggccccgag
gacaagttgcatgagcagtgtcgaccctggaggaagaatgcctgctgttc
taccaacaccagccaggaagcccataaggatgtttcctacctatatagat
tcaactggaaccactgtggagagatggcacctgcctgcaaacggcatttc
atccaggacacctgcctctacgagtgctcccccaacttggggccctggat
ccagcaggtggatcagagctggcgcaaagagcgggtactgaacgtgcccc
tgtgcaaagaggactgtgagcaatggtgggaagattgtcgcacctcctac
acctgcaagagcaactggcacaagggctggaactggacttcagggtttaa
caagtgcgcagtgggagctgcctgccaacctttccatttctacttcccca
cacccactgttctgtgcaatgaaatctggactcactcctacaaggtcagc
aactacagccgagggagtggccgctgcatccagatgtggttcgacccagc
ccagggcaaccccaatgaggaggtggcgaggttctatgctgcagccatga
gtggggctgggccctgggcagcctggcctttcctgcttagcctggcccta
atgctgctgtggctgctcagc

Anti-FOLR1 immunoconjugates contain a cell binding agent linked to a cytotoxin. The cell binding agents can be anti-FOLR1 antibodies or antigen-binding fragments thereof. Examples of therapeutically effective anti-FOLR1 antibodies can be found in US Appl. Pub. No. US 2012/0009181 which is herein incorporated by reference. An example of a therapeutically effective anti-FOLR1 antibody is huMov19 (M9346A) (comprising the sequences of SEQ ID NO:3 and SEQ ID NO:5). The polypeptides of SEQ ID NOs: 3-5 comprise the variable domain of the heavy chain of huMov19 (M9346A), the variable domain light chain version 1.00, and the variable domain light chain version 1.60 of huMov19, respectively. In certain embodiments, the huMov19 anti-FOLR1 antibody is comprised of a variable domain heavy chain represented by SEQ ID NO:3 and a variable domain light chain represented by SEQ ID NO:5 (version 1.60 of huMov19). In certain embodiments, the huMov19 (M9346A) antibody is encoded by the plasmids deposited with the American Type Culture Collection (ATCC), located at 10801 University Boulevard, Manassas, Va. 20110 on Apr. 7, 2010 under the terms of the Budapest Treaty and having ATCC deposit nos. PTA-10772 and PTA-10773 or PTA-10772 and PTA-10774.

Amino acid sequences of huMov19 are provided in Tables 1-4 below:

TABLE 1
Variable heavy chain CDR amino acid sequences

| Antibody | VH-CDR1 | VH-CDR2 | VH-CDR3 |
|---|---|---|---|
| huMov19 | Kabat Defined: | Kabat Defined: | Kabat Defined: |
| (M9346A) | GYFMN (SEQ ID | RIHPYDGDTFYNQKFQG | YDGSRAMDY (SEQ ID |
| | NO: 9) | (SEQ ID NO: 10) | NO: 12) |
| | AbM Defined: | AbM Defined: | AbM Defined: |
| | GYTFTGYFMN | RIHPYDGDTF (SEQ ID | YDGSRAMDY (SEQ ID |
| | (SEQ ID NO: 19) | NO: 11) | NO: 12) |
| muMOV19 | | Kabat Defined | |
| | | RIHPYDGDTFYNQNFKD | |
| | | (SEQ ID NO: 16) | |

TABLE 2
Variable light chain CDR amino acid sequences

| Antibody | VL-CDR1 | VL-CDR2 | VL-CDR3 |
|---|---|---|---|
| huMov19 | KASQSVSFAGTSLMH | RASNLEA (SEQ | QQSREYPYT (SEQ |
| (M9346A) | (SEQ ID NO: 6) | ID NO: 7) | ID NO: 8) |

TABLE 3
Anti-FOLR1 Variable chain amino acid sequences

| FOLR1 | |
|---|---|
| Antibody | Amino Acid Sequence |
| huMov19 - | QVQLVQSGAEVVKPGASVKISCKASGYTFTGYFMNWVKQSPGQSLEWIGRI |
| VH | HPYDGDTFYNQKFQGKATLTVDKSSNTAHMELLSLTSEDFAVYYCTRYDG |
| | SRAMDYWGQGTTVTVSS (SEQ ID NO: 3) |
| huMov19 - | DIVLTQSPLSLAVSLGQPAIISCKASQSVSFAGTSLMHWYHQKPGQQPRLLIY |
| VL | RASNLEAGVPDRFSGSGSKTDFTLNISPVEAEDAATYYCQQSREYPYTFGGG |
| version 1.00 | TKLEIKR (SEQ ID NO: 4) |
| huMOV19- | DIVLTQSPLSLAVSLGQPAIISCKASQSVSFAGTSLMHWYHQKPGQQPRLLIY |
| VL | RASNLEAGVPDRFSGSGSKTDFTLTISPVEAEDAATYYCQQSREYPYTFGGG |
| version 1.60 | TKLEIKR (SEQ ID NO: 5) |

TABLE 4
Full-length heavy and light chain amino acid sequences

| Antibody | Full-Length Amino Acid Sequence |
|---|---|
| huMov19 - | QVQLVQSGAEVVKPGASVKISCKASGYTFTGYFMNWVKQSPGQSLEWIGRI |
| Heavy | HPYDGDTFYNQKFQGKATLTVDKSSNTAHMELLSLTSEDFAVYYCTRYDG |
| | SRAMDYWGQGTTVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSKL |
| | TVDKSRWQQGNVFSCSVMHEALHNHYTQKSLSLSPG (SEQ ID NO: 13) |
| huMov19 - | DIVLTQSPLSLAVSLGQPAIISCKASQSVSFAGTSLMHWYHQKPGQQPRLLIY |
| Light | RASNLEAGVPDRFSGSGSKTDFTLNISPVEAEDAATYYCQQSREYPYTFGGG |
| version 1.00 | TKLEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSPV |
| | TKSFNRGEC (SEQ ID NO: 14) |
| huMOV19- | DIVLTQSPLSLAVSLGQPAIISCKASQSVSFAGTSLMHWYHQKPGQQPRLLIY |
| Light | RASNLEAGVPDRFSGSGSKTDFTLTISPVEAEDAATYYCQQSREYPYTFGGG |
| version 1.60 | TKLEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSPV |
| | TKSFNRGEC (SEQ ID NO: 15) |

In some embodiments, the heavy chain sequence of huMov19 comprises a C-terminal terminal lysine (K) after the last glycine (G) of SEQ ID NO:13 above. In some embodiments, the anti-FOLR1 immunoconjugates comprise humanized antibodies or antigen-binding fragments thereof. In some embodiments, the humanized antibody or fragment is a resurfaced antibody or antigen-binding fragment thereof. In other embodiments, the anti-FOLR1 immunoconjugates comprises a fully human antibody or antigen-binding fragment thereof.

In certain embodiments, the anti-FOLR1 immunoconjugates have one or more of the following effects: inhibit proliferation of tumor cells, reduce the tumorigenicity of a tumor by reducing the frequency of cancer stem cells in the tumor, inhibit tumor growth, increase patient survival, trigger cell death of tumor cells, differentiate tumorigenic cells to a non-tumorigenic state, or prevent or reduce metastasis of tumor cells.

In certain embodiments, the anti-FOLR1 immunoconjugate comprises an antibody that has antibody-dependent cellular cytotoxicity (ADCC) activity.

In some embodiments, the FOLR1 binding molecule is an antibody or antigen-binding fragment comprising the sequences of SEQ ID NOs:6-10 and the sequence of SEQ ID NO:12. In some embodiments, the FOLR1 binding molecule is an antibody or antigen-binding fragment comprising the sequences of SEQ ID NOs:6-9 and the sequences of SEQ ID NOs:11 and 12 In some embodiments, the FOLR1 binding molecule is an antibody or antigen-binding fragment thereof comprising the sequences of SEQ ID NOs: 6-8, 19, 11, and 12.

Also provided are polypeptides that comprise a polypeptide having at least about 90% sequence identity to SEQ ID NO:3, SEQ ID NO:4 or SEQ ID NO:5. In certain embodiments, the polypeptide comprises a polypeptide having at least about 95%, at least about 96%, at least about 97%, at least about 98%, or at least about 99% sequence identity to SEQ ID NO:3, SEQ ID NO:4 or SEQ ID NO:5 Thus, in certain embodiments, the polypeptide comprises (a) a polypeptide having at least about 95% sequence identity to SEQ ID NO:3 and/or (b) a polypeptide having at least about 95% sequence identity to SEQ ID NO:4 or SEQ ID NO:5. In certain embodiments, the polypeptide comprises (a) a polypeptide having the amino acid sequence of SEQ ID NO:3; and/or (b) a polypeptide having the amino acid sequence of SEQ ID NO:4 or SEQ ID NO:5. In certain embodiments, the polypeptide is an antibody and/or the polypeptide specifically binds FOLR1. In certain embodiments, the polypeptide is a murine, chimeric, or humanized antibody that specifically binds FOLR1. In certain embodiments, the polypeptide having a certain percentage of sequence identity to SEQ ID NO:3, SEQ ID NO:4 or SEQ ID NO:5 differs from SEQ ID NO:3, SEQ ID NO:4 or SEQ ID NO:5 by conservative amino acid substitutions only.

Polypeptides can comprise one of the individual light chains or heavy chains described herein. Antibodies and polypeptides can also comprise both a light chain and a heavy chain.

The polypeptides of the present invention can be recombinant polypeptides, natural polypeptides, or synthetic polypeptides comprising an antibody, or fragment thereof, against a human FOLR1 or PD-1.

The polypeptides and analogs can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in REMINGTON'S PHARMACEUTICAL SCIENCES, 20th ed., Mack Publishing Co., Easton, Pa. (2000).

Methods known in the art for purifying antibodies and other proteins also include, for example, those described in U.S. Patent Publication No. 2008/0312425, 2008/0177048, and 2009/0187005, each of which is hereby incorporated by reference herein in its entirety.

Suitable drugs or prodrugs are known in the art. The drugs or prodrugs can be cytotoxic agents. The cytotoxic agent used in the cytotoxic conjugate of the present invention can be any compound that results in the death of a cell, or induces cell death, or in some manner decreases cell viability, and includes, for example, maytansinoids and maytansinoid analogs.

Such conjugates can be prepared by using a linking group in order to link a drug or prodrug to the antibody or functional equivalent. Suitable linking groups are well known in the art and include, for example, disulfide groups, thioether groups, acid labile groups, photolabile groups, peptidase labile groups and esterase labile groups.

The drug or prodrug can, for example, be linked to the anti-FOLR1 antibody or fragment thereof through a disulfide bond. The linker molecule or crosslinking agent comprises a reactive chemical group that can react with the anti-FOLR1 antibody or fragment thereof. The reactive chemical groups for reaction with the cell-binding agent can be N-succinimidyl esters and N-sulfosuccinimidyl esters. Additionally the linker molecule comprises a reactive chemical group, which can be a dithiopyridyl group that can react with the drug to form a disulfide bond. Linker molecules include, for example, N-succinimidyl 4-(2-pyridyldithio)2-sulfobutanoate (sulfo-SPDB) (see US Publication No. 20090274713). For example, the antibody or cell binding agent can be modified with crosslinking reagents and the antibody or cell binding agent containing free or protected thiol groups thus derived is then reacted with a disulfide- or thiol-containing maytansinoid to produce conjugates. The conjugates can be purified by chromatography, including but not limited to HPLC, size-exclusion, adsorption, ion exchange and affinity capture, dialysis or tangential flow filtration.

In another aspect of the present invention, the anti-FOLR1 antibody is linked to cytotoxic drugs via disulfide bonds and a polyethylene glycol spacer in enhancing the potency, solubility or the efficacy of the immunoconjugate. Such cleavable hydrophilic linkers are described in WO2009/0134976. The additional benefit of this linker design is the desired high monomer ratio and the minimal aggregation of the antibody-drug conjugate. Specifically contemplated in this aspect are conjugates of cell-binding agents and drugs linked via disulfide group (—S—S—) bearing polyethylene glycol spacers ((CH₂CH₂O)ₙ₌₁₋₁₄) with a narrow range of drug load of 2-8 are described that show relatively high potent biological activity toward cancer cells and have the desired biochemical properties of high conjugation yield and high monomer ratio with minimal protein aggregation.

In some embodiments, the linker is a linker containing at least one charged group as described, for example, in U.S. Patent Publication No. 2012/0282282, the contents of which are entirely incorporated herein by reference. In some embodiments, the charged or pro-charged cross-linkers are those containing sulfonate, phosphate, carboxyl or quaternary amine substituents that significantly increase the solubility of the modified cell-binding agent and the cell-binding agent-drug conjugates, especially for monoclonal antibody-drug conjugates with 2 to 20 drugs/antibody linked. Conjugates prepared from linkers containing a pro-charged moiety would produce one or more charged moieties after the conjugate is metabolized in a cell. In some embodiments, the linker is selected from the group consisting of: N-succinimidyl 4-(2-pyridyldithio)-2-sulfopentanoate (sulfo-SPP) and N-succinimidyl 4-(2-pyridyldithio)-2-sulfobutanoate (sulfo-SPDB).

Many of the linkers disclosed herein are described in detail in U.S. Patent Publication Nos. 2005/0169933, 2009/0274713, and 2012/0282282, and in WO2009/0134976 and WO2012/135675; the contents of which are entirely incorporated herein by reference.

The present invention includes aspects wherein about 2 to about 8 drug molecules (“drug load”), for example, maytansinoid, are linked to an anti-FOLR1 antibody or fragment thereof. “Drug load”, as used herein, refers to the number of drug molecules (e.g., a maytansinoid) that can be attached to a cell binding agent (e.g., an anti-FOLR1 antibody or fragment thereof). In one aspect, the number of drug molecules that can be attached to a cell binding agent can average from about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1). N2′-deacetyl-N2′-(3-mercapto-1-oxopropyl)-maytansine (DM1) and N2′-deacetyl-N2′-(4-mercapto-4-methyl-1-oxopentyl) maytansine (DM4) can be used.

Further, in some embodiements, the maytansinoid (e.g., DM4) is linked (e.g., by sulfo-SPDB) to the anti-FOLR1 antibody or antigen-binding fragment thereof via a lysine residue of the antibody or antigen-binding fragment thereof. In some embodiements, 1-10 maytansinoids (e.g., DM4) are linked (e.g., by sulfo-SPDB) to the anti-FOLR1 antibody or antigen-binding fragment thereof via 1-10 lysine residues of the antibody or antigen-binding fragment thereof. In some embodiements, 2-8 maytansinoids (e.g., DM4) are linked (e.g., by sulfo-SPDB) to the anti-FOLR1 antibody or antigen-binding fragment thereof via 2-8 lysine residues of the antibody or antigen-binding fragment thereof. In some embodiements, 2-5 maytansinoids (e.g., DM4) are linked (e.g., by sulfo-SPDB) to the anti-FOLR1 antibody or antigen-binding fragment thereof via 2-5 lysine residues of the antibody or antigen-binding fragment thereof. In some embodiements, 3-4 maytansinoids (e.g., DM4) are linked (e.g., by sulfo-SPDB) to the anti-FOLR1 antibody or antigen-binding fragment thereof via 3-4 lysine residues of the antibody or antigen-binding fragment thereof.

Further, in one aspect, an immunoconjugate comprises 1 maytansinoid per antibody. In another aspect, an immunoconjugate comprises 2 maytansinoids per antibody. In another aspect, an immunoconjugate comprises 3 maytansinoids per antibody. In another aspect, an immunoconjugate comprises 4 maytansinoids per antibody. In another aspect, an immunoconjugate comprises 5 maytansinoids per antibody. In another aspect, an immunoconjugate comprises 6 maytansinoids per antibody. In another aspect, an immunoconjugate comprises 7 maytansinoids per antibody. In another aspect, an immunoconjugate comprises 8 maytansinoids per antibody.

In one aspect, an immunoconjugate (e.g., an immunoconjugate comprising the linker sulfo-SPDB and the maytansinoid DM4) comprises about 1 to about 8 maytansinoids per antibody. In another aspect, an immunoconjugate (e.g., an immunoconjugate comprising the linker sulfo-SPDB and the maytansinoid DM4) comprises about 2 to about 7 maytansinoids per antibody. In another aspect, an immunoconjugate (e.g., an immunoconjugate comprising the linker sulfo-SPDB and the maytansinoid DM4) comprises about 2 to about 6 maytansinoids per antibody. In another aspect, an immunoconjugate (e.g., an immunoconjugate comprising the linker sulfo-SPDB and the maytansinoid DM4) comprises about 2 to about 5 maytansinoids per antibody. In another aspect, an immunoconjugate (e.g., an immunoconjugate comprising the linker sulfo-SPDB and the maytansinoid DM4) comprises about 3 to about 5 maytansinoids per antibody. In another aspect, an immunoconjugate (e.g., an immunoconjugate comprising the linker sulfo-SPDB and the maytansinoid DM4) comprises about 3 to about 4 maytansinoids per antibody.

In one aspect, a composition comprising immunoconjugates has an average of about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1) drug molecules (e.g., maytansinoids) attached per antibody. In one aspect, a composition comprising immunoconjugates has an average of about 1 to about 8 drug molecules (e.g., maytansinoids) per antibody. In one aspect, a composition comprising immunoconjugates has an average of about 2 to about 7 drug molecules (e.g., maytansinoids) per antibody. In one aspect, a composition comprising immunoconjugates has an average of about 2 to about 6 drug molecules (e.g., maytansinoids) per antibody. In one aspect, a composition comprising immunoconjugates has an average of about 2 to about 5 drug molecules (e.g., maytansinoids) per antibody. In one aspect, a composition comprising immunoconjugates has an average of about 3 to about 5 drug molecules (e.g., maytansinoids) per antibody. In one aspect, a composition comprising immunoconjugates has an average of about 3 to about 4 drug molecules (e.g., maytansinoids) per antibody.

In one aspect, a composition comprising immunoconjugates has an average of about 2±0.5, about 3±0.5, about 4±0.5, about 5±0.5, about 6±0.5, about 7±0.5, or about 8±0.5 drug molecules (e.g., maytansinoids) attached per antibody. In one aspect, a composition comprising immunoconjugates has an average of about 3.5±0.5 drug molecules (e.g., maytansinoids) per antibody.

The anti-FOLR1 antibody or fragment thereof can be modified by reacting a bifunctional crosslinking reagent with the anti-FOLR1 antibody or fragment thereof, thereby resulting in the covalent attachment of a linker molecule to the anti-FOLR1 antibody or fragment thereof. As used herein, a “bifunctional crosslinking reagent” is any chemical moiety that covalently links a cell-binding agent to a drug, such as the drugs described herein. In another method, a portion of the linking moiety is provided by the drug. In this respect, the drug comprises a linking moiety that is part of a larger linker molecule that is used to join the cell-binding agent to the drug. For example, to form the maytansinoid DM1 or DM4, the side chain at the C-3 hydroxyl group of maytansine is modified to have a free sulfhydryl group (SH). This thiolated form of maytansine can react with a modified cell-binding agent to form a conjugate. Therefore, the final linker is assembled from two components, one of which is provided by the crosslinking reagent, while the other is provided by the side chain from DM1 or DM4.

The drug molecules can also be linked to the antibody molecules through an intermediary carrier molecule such as serum albumin.

As used herein, the expression “linked to a cell-binding agent” or “linked to an anti-FOLR1 antibody or fragment” refers to the conjugate molecule comprising at least one drug derivative bound to a cell-binding agent, anti-FOLR1 antibody, or fragment via a suitable linking group, or a precursor thereof. Exemplary linking groups are SPDB or sulfo-SPDB.

In certain embodiments, cytotoxic agents useful in the present invention are maytansinoids and maytansinoid analogs. Examples of suitable maytansinoids include esters of maytansinol and maytansinol analogs. Included are any drugs that inhibit microtubule formation and that are highly toxic to mammalian cells, as are maytansinol and maytansinol analogs.

Examples of suitable maytansinol esters include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 7,276,497 and 7,473,796.

In a certain embodiment, the immunoconjugates of the invention utilize the thiol-containing maytansinoid (DM1), formally termed N^{2′}-deacetyl-N^{2′}-(3-mercapto-1-oxopropyl)-maytansine, as the cytotoxic agent. DM1 is represented by the following structural formula (I):

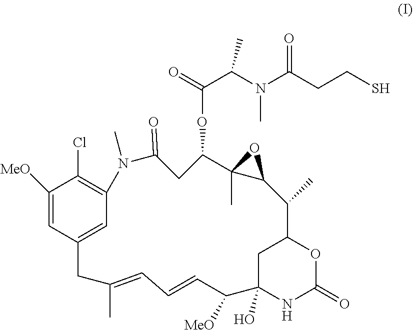 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present invention utilize the thiol-containing maytansinoid N^{2′}-deacetyl-N^{2′}(4-methyl-4-mercapto-1-oxopentyl)-maytansine (e.g., DM4) as the cytotoxic agent. DM4 is represented by the following structural formula (II):

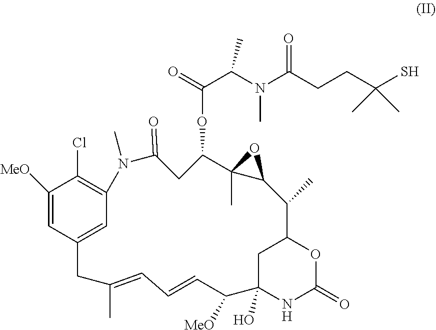 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N^{2′}-deacetyl-N-^{2′}(4-mercapto-1-oxopentyl)-maytansine (termed DM3), represented by the following structural formula (III):

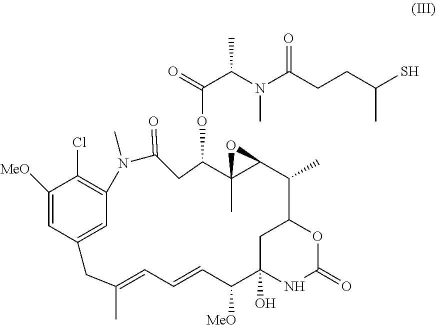 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugate of the present invention. In this regard, the entire disclosure of U.S. Pat. Nos. 5,208,020 and 7,276,697 is incorporated herein by reference.

Many positions on maytansinoids can serve as the position to chemically link the linking moiety. For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to chemically link the linking moiety, and in some particular embodiments, the C-3 position of maytansinol serves as the position to chemically link the linking moiety.

Structural representations of some conjugates are shown below:

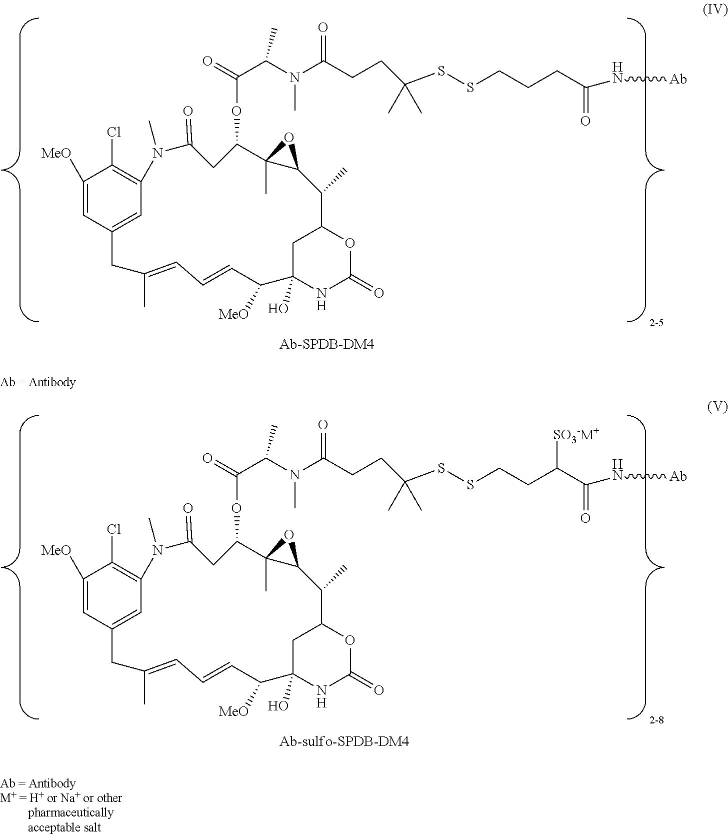

In some embodiments, in fomula (V), M+ is H.

Also included in the present invention are any stereoisomers and mixtures thereof for any compounds or conjugates depicted by any structures above.

Several descriptions for producing such antibody-maytansinoid conjugates are provided in U.S. Pat. Nos. 6,333,410, 6,441,163, 6,716,821, and 7,368,565, each of which is incorporated herein in its entirety.

In general, a solution of an antibody in aqueous buffer can be incubated with a molar excess of maytansinoids having a disulfide moiety that bears a reactive group. The reaction mixture can be quenched by addition of excess amine (such as ethanolamine, taurine, etc.). The maytansinoid-antibody conjugate can then be purified by gel filtration.

The number of maytansinoid molecules bound per antibody molecule can be determined by measuring spectrophotometrically the ratio of the absorbance at 252 nm and 280 nm. The average number of maytansinoid molecules/antibody can be, for example, 1-10 or 2-5. The average number of maytansinoid molecules/antibody can be, for example about 3 to about 4. The average number of maytansinoid molecules/antibody can be about 3.5 or 3.5+/−0.5.

Conjugates of antibodies with maytansinoid or other drugs can be evaluated for their ability to suppress proliferation of various unwanted cell lines in vitro. For example, cell lines such as the human lymphoma cell line Daudi and the human lymphoma cell line Ramos, can easily be used for the assessment of cytotoxicity of these compounds. Cells to be evaluated can be exposed to the compounds for 4 to 5 days and the surviving fractions of cells measured in direct assays by known methods. IC₅₀ values can then be calculated from the results of the assays.

The immunoconjugates can, according to some embodiments described herein, be internalized into cells. The immunoconjugate, therefore, can exert a therapeutic effect when it is taken up by, or internalized, by an FOLR1-expressing cell. In some particular embodiments, the immunoconjugate comprises an antibody, antibody fragment, or polypeptide, linked to a cytotoxic agent by a cleavable linker, and the cytotoxic agent is cleaved from the antibody, antibody fragment, or polypeptide, wherein it is internalized by an FOLR1-expressing cell.

In some embodiments, the immunoconjugates are capable of reducing tumor volume. For example, in some embodiments, treatment with an immunoconjugate results in a % T/C value that is less than about 50%, less than about 45%, less than about 40%, less than about 35%, less than about 30%, less than about 25%, less than about 20%, less than about 15%, less than about 10%, or less than about 5%. In some particular embodiments, the immunoconjugates can reduce tumor size in a OVCAR-3, IGROV-1, and/or OV-90 xenograft model. In some embodiments, the immunoconjugates are capable of inhibiting metastases.

### III. Anti-PD-1 Antibodies

Described herein are methods of administering anti-FOLR1 immunoconjugates such as IMGN853 in combination with anti-PD-1 antibodies or antigen-binding fragments thereof (e.g., pembrolizumab).

In certain embodiments, the anti-PD-1 antibody or antigen-binding fragment thereof (e.g., pembrolizumab) is capable of inhibiting tumor growth. In certain embodiments, the anti-PD-1 antibody or antigen-binding fragment thereof (e.g., pembrolizumab) is capable of inhibiting tumor growth in vivo (e.g., in a xenograft mouse model and/or in a human having cancer). In certain embodiments, the anti-PD-1 antibody or antigen-binding fragment thereof (e.g., pembrolizumab) is capable of inhibiting angiogenesis.

A full-length amino acid sequence for PD-1 is provided at UniProtKB Accession No. Q15116 and herein as SEQ ID NO:17:

(SEQ ID NO: 17)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL,
(SEQ ID NO: 18)
the signal sequence of which is MQIPQAPWPVVWAVLQLG
WR.

Thus, in some embodiments, an anti-PD-1 antibody or antigen-binding fragment thereof binds to an epitope in SEQ ID NO: 17 or to an epitope in the mature version of SEQ ID NO: 17 (i.e., SEQ ID NO:17 lacking the signal sequence.)

Anti-PD-1 antibodies and antigen-binding fragments thereof can comprise polypeptides comprising the variable light chains or variable heavy chains described herein. Anti-PD-1 antibodies and polypeptides can also comprise both a variable light chain and a variable heavy chain. Anti-PD-1 antibodies, and the variable light chains and variable heavy chains thereof, are described in at least U.S. Pat. No. 8,354,509 and U.S. Pat. No. 8,900,587, each of which is incorporated herein by reference in its entirety.

In some embodiments, an anti-PD-1 antibody is pembrolizumab (Keytruda®). In some embodiments, an anti-PD-1 antibody or antigen-binding fragment thereof comprises the CDR, VH, VL, or VH and VL sequences of pembrolizumab. Amino acid sequences of pembrolizumab are provided in Tables 5-8 below:

TABLE 5
Pembrolizumab variable heavy chain CDR amino acid
sequences

| Antibody | VH-CDR1 | VH-CDR2 | VH-CDR3 |
|---|---|---|---|
| Pembrolizumab | NYYMY | GINPSNGGTNENEKEKN | RDYRFDMGFDY |
| | (SEQ ID | (SEQ ID NO: 21) | (SEQ ID NO: |
| | NO: 20) | | 22) |

TABLE 6
Pembrolizumab variable light chain CDR amino acid
sequences

| Antibody | VL-CDR1 | VL-CDR2 | VL-CDR3 |
|---|---|---|---|
| Pembrolizumab | RASKGVSTSGYSYLH | LASYLES | QHSRDLPLT |
| | (SEQ ID NO: 23) | (SEQ ID | (SEQ ID |
| | | NO: 24) | NO: 25) |

TABLE 7
Pembrolizumab variable chain amino acid sequences

| Pembrolizumab | Amino Acid Sequence |
|---|---|
| VH | QVQLVQSGVEVKKPGASVKVSCKASGYTFTNYYMYWVRQAPGQGLE |
| | WMGGINPSNGGTNFNEKFKNRVTLTTDSSTTTAYMELKSLQFDDTAVY |
| | YCARRDYRFDMGFDYWGQGTTVTVSS (SEQ ID NO: 26) |
| VL | EIVLTQSPATLSLSPGERATLSCRASKGVSTSGYSYLHWYQQKPGQAPR |
| | LLIYLASYLESGVPARFSGSGSGTDFTLTISSLEPEDFAVYYCQHSRDLPL |
| | TFGGGTKVEIK (SEQ ID NO: 27) |

TABLE 8
Pembrolizumab full-length heavy and light chain amino acid sequences

| Pembrolizumab | Full-Length Amino Acid Sequence |
|---|---|
| Heavy | QVQLVQSGVEVKKPGASVKVSCKASGYTFTNYYMYWVRQAPGQGLE |
| | WMGGINPSNGGTNENEKFKNRVTLTTDSSTTTAYMELKSLQFDDTAVY |
| | YCARRDYRFDMGFDYWGQGTTVTVSSASTKGPSVFPLAPCSRSTSESTA |
| | ALGCLVKDYFPEPVTVSWNSGALTSGVHTFPAVLQSSGLYSLSSVVTVP |
| | SSSLGTKTYTCNVDHKPSNTKVDKRVESKYGPPCPPCPAPEFLGGPSVFL |
| | FPPKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTISKA |
| | KGQPREPQVYTLPPSQEEMTKNQVSLTCLVKGFYPSDIAVEWESNGQPE |
| | NNYKTTPPVLDSDGSFFLYSRLTVDKSRWQEGNVFSCSVMHEALHNHY |
| | TQKSLSLSLGK (SEQ ID NO: 28) |
| Light | EIVLTQSPATLSLSPGERATLSCRASKGVSTSGYSYLHWYQQKPGQAPR |
| | LLIYLASYLESGVPARFSGSGSGTDFTLTISSLEPEDFAVYYCQHSRDLPL |
| | TFGGGTKVEIKRTVAAPSVFIFPPSDEQLKSGTASVVCLLNNFYPREAKV |
| | QWKVDNALQSGNSQESVTEQDSKDSTYSLSSTLTLSKADYEKHKVYAC |
| | EVTHQGLSSPVTKSFNRGEC (SEQ ID NO: 29) |

In some embodiments, an anti-PD-1 antibody or antigen-binding fragment thereof comprises the CDR sequences of pembrolizumab (i.e. SEQ ID NO: 20, 21, 22, 23, 24, and 25) and blocks the interaction between PD-1 and PD-L1. In further embodiments, an anti-PD-1 antibody or antigen-binding fragment thereof comprises the VH, VL, or VH and VL sequences of pembrolizumab and blocks the interaction between PD-1 and PD-L1. In some embodiments, an anti-PD-1 antibody or antigen-binding fragment thereof comprises the CDR sequences of pembrolizumab and blocks the interaction between PD-1 and PD-L2. In further embodiments, an anti-PD-1 antibody or antigen-binding fragment thereof comprises the, VH, VL, or VH and VL sequences of pembrolizumab and blocks the interaction between PD-1 and PD-L2. In some embodiments, an anti-PD-1 antibody or antigen-binding fragment thereof comprises the CDR, VH, VL, or VH and VL sequences of pembrolizumab, blocks the interaction between PD-1 and PD-L1, and blocks the interaction between PD-1 and PD-L2. In some embodiments, an anti-PD-1 antibody or antigen-binding fragment thereof comprises the CDR, VH, VL, or VH and VL sequences of pembrolizumab and reduces or decreases inhibition of T cell proliferation and/or cytokine production. In some embodiments, an anti-PD-1 antibody or antigen-binding fragment thereof comprises the CDR sequences or the, VH, VL, or VH and VL sequences of pembrolizumab and releases PD-1 pathway-mediated inhibition of an immune response, e.g., an anti-tumor immune response.

### IV. Pharmaceutical Compositions and Kits

As provided herein, anti-FOLR1 immunoconjugates (e.g., IMGN853) can be used in combination with an anti-PD-1 antibody or antigen-binding fragment thereof (e.g., pembrolizumab) to treat cancer. The cancer can be ovarian cancer, peritoneal cancer, or fallopian tube cancer. In some instances, the cancer can be endometrial cancer.

In some embodiments, an anti-FOLR1 immunoconjugate (e.g., IMGN853) and an anti-PD-1 antibody or antigen-binding fragment thereof (e.g., pembrolizumab) are contained within the same pharmaceutical composition. In some embodiments, an anti-FOLR1 immunoconjugate (e.g., IMGN853) and an anti-PD-1 antibody or antigen-binding fragment thereof (e.g., pembrolizumab) are contained within two separate pharmaceutical compositions within a single kit. In other embodiments, a kit comprises an anti-FOLR1 immunoconjugate (e.g., IMGN853) and instructions to administer the anti-FOLR1 immunoconjugate (e.g., IMGN853) and an anti-PD-1 antibody or antigen-binding fragment thereof (e.g., pembrolizumab). In other embodiments, a kit comprises an anti-PD-1 antibody or antigen-binding fragment thereof (e.g., pembrolizumab) and instructions to administer the anti-PD-1 antibody or antigen-binding fragment thereof (e.g., pembrolizumab) and an anti-FOLR1 immunoconjugate (e.g., IMGN853).

In certain embodiments, the pharmaceutical compositions provided herein comprise an anti-FOLR1 immunoconjugate (e.g., IMGN853) and/or an anti-PD-1 antibody or antigen-binding fragment thereof (e.g., pembrolizumab) and a pharmaceutically acceptable vehicle. In certain embodiments, the pharmaceutical compositions further comprise a preservative. These pharmaceutical compositions find use in inhibiting tumor growth and treating cancer in human patients.

The pharmaceutical compositions for use as provided herein can be administered in any number of ways, for example, by parenteral administration including intravenous, intraarterial, subcutaneous, intraperitoneal or intramuscular injection or infusion. In some embodiments, the pharmaceutical composition is formulated for intravenous (i.v.) administration. In some embodiments, the pharmaceutical composition is formulated for intraperitoneal (i.p.) administration. In some embodiments, the anti-FOLR1 immunoconjugate (IMGN853) and the anti-PD-1 antibody or antigen-binding fragment thereof (e.g., pembrolizumab) are administered intravenously.

### V. Methods of Use

As provided herein, anti-FOLR1 immunoconjugates (e.g., IMGN853) can be used in combination with an anti-PD-1 antibody or antigen-binding fragment thereof (e.g., pembrolizumab) to treat cancer.

### V.A. Cancer Selection

Cancers that can be treated by the methods provided herein include ovarian cancer, peritoneal cancer, and fallopian tube cancer. Endometrial cancer can also be treated by the methods provided herein. The cancer can be a primary or metastatic cancer.

More particular examples of such cancers include ovarian epithelial ovarian cancer, ovarian primary peritoneal cancer, or ovarian fallopian tube cancer. In some embodiments, the subject has previously untreated ovarian cancer. In other embodiments, the subject has previously treated ovarian cancer (e.g., previously treated with a platinum compound, a taxane, bevacizumab, a PARP inhibitor, or a combination thereof). In some embodiments, the subject has platinum sensitive recurrent epithelial ovarian, primary peritoneal, or fallopian tube cancer. In other embodiments, the subject has platinum resistant recurrent epithelial ovarian, primary peritoneal, or fallopian tube cancer.

In certain embodiments, the cancer is ovarian, peritoneal, or fallopian tube cancer. The combination of an anti-FOLR1 immunoconjugate (e.g., IMGN853) and an anti-PD-1 antibody or antigen-binding fragment thereof (e.g., pembrolizumab) can be administered to treat an ovarian, peritoneal, or fallopian tube cancer as a first-line therapy, a second-line therapy, a third-line therapy, or a fourth or later-line therapy. The combination of an anti-FOLR1 immunoconjugate (e.g., IMGN853) and an anti-PD1 agent (e.g., pembrolizumab) can be administered to treat an ovarian, peritoneal, or fallopian tube cancer as an adjuvant therapy, neoadjuvant therapy, or maintenance therapy.

In certain embodiments, the cancer is endometrial cancer. The combination of an anti-FOLR1 immunoconjugate (e.g., IMGN853) and an anti-PD-1 antibody or antigen-binding fragment thereof (e.g., pembrolizumab) can be administered to treat an endometrial cancer as a first-line therapy, a second-line therapy, a third-line therapy, or a fourth or later-line therapy. The combination of an anti-FOLR1 immunoconjugate (e.g., IMGN853) and an anti-PD1 agent (e.g., pembrolizumab) can be administered to treat an endometrial cancer as an adjuvant therapy, neoadjuvant therapy, or maintenance therapy.

In certain embodiments, the cancer is serous endometrial cancer. The combination of an anti-FOLR1 immunoconjugate (e.g., IMGN853) and an anti-PD-1 antibody or antigen-binding fragment thereof (e.g., pembrolizumab) can be administered to treat a serous endometrial cancer as a first-line therapy, a second-line therapy, a third-line therapy, or a fourth or later-line therapy. The combination of an anti-FOLR1 immunoconjugate (e.g., IMGN853) and an anti-PD1 agent (e.g., pembrolizumab) can be administered to treat a serous endometrial cancer as an adjuvant therapy, neoadjuvant therapy, or maintenance therapy.

In certain embodiments, the cancer is endometrioid endometrial cancer. The combination of an anti-FOLR1 immunoconjugate (e.g., IMGN853) and an anti-PD-1 antibody or antigen-binding fragment thereof (e.g., pembrolizumab) can be administered to treat an endometrioid endometrial cancer as a first-line therapy, a second-line therapy, a third-line therapy, or a fourth or later-line therapy. The combination of an anti-FOLR1 immunoconjugate (e.g., IMGN853) and an anti-PD1 agent (e.g., pembrolizumab) can be administered to treat an endometrioid endometrial cancer as an adjuvant therapy, neoadjuvant therapy, or maintenance therapy.

In certain embodiments, the cancer is ovarian cancer. In certain embodiments, the ovarian cancer is epithelial ovarian cancer (EOC). In certain embodiments, the ovarian cancer (e.g., an EOC) is platinum resistant, relapsed, or refractory or platinum sensitive. The combination of an anti-FOLR1 immunoconjugate (e.g., IMGN853) and an anti-PD-1 antibody or antigen-binding fragment thereof (e.g., pembrolizumab) can be administered to an EOC, e.g., an EOC that is platinum resistant, relapsed, or refractory as a first-line therapy, a second-line therapy, a third-line therapy, or a fourth or later-line therapy. The combination of an anti-FOLR1 immunoconjugate (e.g., IMGN853) and an anti-PD-1 antibody or antigen-binding fragment thereof (e.g., pembrolizumab) can be administered to an EOC, e.g., an EOC that is platinum resistant, relapsed, or refractory as an adjuvant therapy, neoadjuvant therapy, or a maintenance therapy.

In certain embodiments, the cancer is peritoneal cancer. In certain embodiments, the peritoneal cancer is primary peritoneal cancer. The combination of an anti-FOLR1 immunoconjugate (e.g., IMGN853) and an anti-PD-1 antibody or antigen-binding fragment thereof (e.g., pembrolizumab) can be administered to a primary peritoneal cancer as a first-line therapy, a second-line therapy, a third-line therapy, or a fourth or later-line therapy. The combination of an anti-FOLR1 immunoconjugate (e.g., IMGN853) and an anti-PD-1 antibody or antigen-binding fragment thereof (e.g., pembrolizumab) can be administered to a primary peritoneal cancer as an adjuvant therapy, a neoadjuvant therapy, or a maintenance therapy.

In certain embodiments, the cancer is platinum refractory. In certain embodiments, the cancer is primary platinum refractory. The combination of an anti-FOLR1 immunoconjugate (e.g., IMGN853) and an anti-PD-1 antibody or antigen-binding fragment thereof (e.g., pembrolizumab) can be administered to a platinum refractory cancer or a primary platinum refractory cancer as a first-line therapy, a second-line therapy, a third-line therapy, or a fourth or greater-line therapy. The combination of an anti-FOLR1 immunoconjugate (e.g., IMGN853) and an anti-PD-1 antibody or antigen-binding fragment thereof (e.g., pembrolizumab) can be administered to a platinum refractory cancer or a primary platinum refractory cancer as an adjuvant therapy, a neoadjuvant therapy, or a maintenance therapy.

In certain embodiments, the cancer is platinum sensitive. The combination of an anti-FOLR1 immunoconjugate (e.g., IMGN853) and an anti-PD-1 antibody or antigen-binding fragment thereof (e.g., pembrolizumab) can be administered to a platinum sensitive cancer as a first-line therapy, a second-line therapy, a third-line therapy, or a fourth or later-line therapy.

The combination of an anti-FOLR1 immunoconjugate (e.g., IMGN853) and an anti-PD-1 antibody or antigen-binding fragment thereof (e.g., pembrolizumab) can be administered to a platinum sensitive cancer as an adjuvant therapy, a neoadjuvant therapy, or a maintenance therapy.

In certain embodiments, the cancer is a metastatic or advanced cancer. The combination of an anti-FOLR1 immunoconjugate (e.g., IMGN853) and an anti-PD-1 antibody or antigen-binding fragment thereof (e.g., pembrolizumab) can be administered to a metastatic or advanced cancer as a first-line therapy, a second-line therapy, a third-line therapy, or a fourth or greater-line therapy. The combination of an anti-FOLR1 immunoconjugate (e.g., IMGN853) and an anti-PD-1 antibody or antigen-binding fragment thereof (e.g., pembrolizumab) can be administered to a metastatic or advanced cancer as an adjuvant therapy, a neoadjuvant therapy, or a maintenance therapy.

Administration of the combination of an anti-FOLR1 immunoconjugate (e.g., IMGN853) and an anti-PD-1 antibody or antigen-binding fragment thereof (e.g., pembrolizumab) as a “second-line” therapy includes administration wherein the first-line therapy was, for example, administration of a single agent, administration of a combination of agents, surgery, radiation, or a combination thereof. Administration of the combination of an anti-FOLR1 immunoconjugate (e.g., IMGN853) and an anti-PD-1 antibody or antigen-binding fragment thereof (e.g., pembrolizumab) as a “third-line” therapy includes administration wherein the first-line therapy was, for example, administration of a single agent, administration of a combination of agents, surgery, radiation, or a combination thereof and wherein the second-line therapy was, for example, administration of a single agent, administration of a combination of agents, surgery, radiation, or a combination thereof. Thus, administration of the combination of an anti-FOLR1 immunoconjugate (e.g., IMGN853) and an anti-PD-1 antibody or antigen-binding fragment thereof (e.g., pembrolizumab) as a “third-line” therapy includes for example, administration following a first-line therapy that was administration of a single agent, and a second-line therapy that was administration of a combination of agents. Administration of the combination of an anti-FOLR1 immunoconjugate (e.g., IMGN853) and an anti-PD-1 antibody or antigen-binding fragment thereof (e.g., pembrolizumab) as a “third-line” therapy also includes, for example, administration following a first-line therapy that was administration of a combination of agents, and a second-line therapy that was administration of a single agent. Administration of the combination of an anti-FOLR1 immunoconjugate (e.g., IMGN853) and an anti-PD-1 antibody or antigen-binding fragment thereof (e.g., pembrolizumab) also includes, for example, administration following a first-line therapy that was administration of a combination of agents, and a second-line therapy that was administration of a combination of agents. Administration of the combination of an anti-FOLR1 immunoconjugate (e.g., IMGN853) and an anti-PD-1 antibody or antigen-binding fragment thereof (e.g., pembrolizumab) as a “third-line” therapy also includes, for example, administration following a first-line therapy that was administration of a combination of agents and a surgery, and a second-line therapy that was administration of a combination of agents. In some embodiments, a patient receiving administration of the combination of ananti-FOLR1 immunoconjugate (e.g., IMGN853) and an anti-PD-1 antibody or antigen-binding fragment thereof (e.g., pembrolizumab) has had 1, 2, 3, 4, or more lines of of therapy. In some embodiments, a patient receiving administration of the combination of ananti-FOLR1 immunoconjugate (e.g., IMGN853) and an anti-PD-1 antibody or antigen-binding fragment thereof (e.g., pembrolizumab) has had no more than 1, 2, 3, or 4 lines of of therapy.

### V.B. Scoring Systems

Three different scoring systems are described herein without limitation. Other scoring systems besides the examples provided herein may be utilized according to the implementations disclosed. As applied herein, the description of a score as “moderate” or “medium,” for example, only applies in the context of the scoring system utilized. For example, a sample scored according to heterogenous/homogenous scoring system may be described as “moderate,” but not “medium FRα” according to the H score system. The term “moderate” is not applied in the context of the H scoring system. A sample may be evaluated by more than one scoring system, and there may be overlap in the labeling of that sample. For example, a sample described as “moderate” according to the heterogenous/homogenous scoring system may also be separately scored as “medium” according to the H score system.

H Scoring Sytem. Wet tissue, tumor blocks, and unstained slides may be obtained. In some instances, unstained, serially-cut, 4-5 microns thick sections may be placed on Superfrost® PLUS slides and obtained from study sites. If a paraffin block is received, six (6) slides may be prepared. If either a paraffin block or a wet tissue (which is then processed into a paraffin block for retrospective study) is received, three (3) slides may be prepared. Specimens may be automatically processed for staining once all accessioning and quality checks have been completed. Slides may be stained with FOLR1 negative and FOLR1 positive markers. In the event that one of the FOLR1 markers fails, a repeat test may be performed. The repeat test may test for the positive and negative markers and may use different available positive and negative FOLR1 markers. A negative marker control for each specimen may be evaluated for acceptable signal/noise ratio and background staining. The negative control may be scored and an assessment of accept or reject can be made. The positive biomarker may be assessed for evaluability based on tissue and cell viability, morphology, and the presence of discernable background staining.

Immunological detection (by immunohistochemistry) of FOLR1 can be scored using H-scores. The percentage of cells staining at each intensity in all relevant cellular compartments (e.g., membrane and cytoplasm), may be obtained. H-scores combine staining intensity scores (e.g., a score of 0 to 3, wherein 0 represents no staining, and 3 represents strong staining) with the percentage of cells that are positive for membrane staining (i.e., uniformity). An H-score can be calculated as follows:

H score=[0*(percentage of cells staining at intensity 0)]+[1*(percentage of cells staining at intensity 1)]+[2*(percentage of cells staining at intensity 2)]+[3*(percentage of cells staining at intensity 3)].

Accordingly, an H-score can range from 0 (no cell membranes staining) to 300 (all cell membranes staining at intensity 3).

Expression of FRα may be defined as “low”, “medium”, or “high” based upon a binned scoring algorithm. “Low expression” refers to a range of at least 25% of cells to 49% of cells in the sample obtained from the patient having an IHC score of 2 or 3. “Medium expression” refers to a range of at least 50% of cells to 74% of cells in the sample obtained from the patient having an IHC score of 2 or 3. “High expression” refers to a range of 75% or more cells in the sample obtained from the patient having an IHC score of 2 or 3.

IHC Visual Scoring System. Wet tissue, tumor blocks, and unstained slides may be obtained. In some instances, unstained, serially-cut, 4-5 microns thick sections may be placed on Superfrost® PLUS slides and obtained from study sites. If a paraffin block is received, 6 slides may be prepared. If either a paraffin block or a wet tissue (which is then processed into a paraffin block for retrospective study) is received, three (3) slides may be prepared. Specimens may be automatically processed for staining once all accessioning and quality checks have been completed. Slides may be stained with FOLR1 negative and FOLR1 positive markers. In the event that one of the FOLR1 markers fails, a repeat test may be performed. The repeat test may test for the positive and negative markers. A negative marker control for each specimen may be evaluated for acceptable signal/noise ratio and background staining. The negative control may be scored and an assessment of accept or reject can be made. The positive biomarker may be assessed for evaluability based on tissue and cell viability, morphology, and the presence of discernable background staining.

The FOLR1 protein expression can also be measured by immunohistochemistry (IHC). The percentage of cells staining in all relevant cellular compartments (e.g., membrane and cytoplasm), may be obtained. The FOLR1 biomarker slides may be assessed for tumor cell membrane staining per the scoring algorithm provided in Table 9 below:

TABLE 9
Scoring algorithm

| | |
|---|---|
| Staining Pattern | Stratification Score |

Step 1: Evaluation at the 50% cutoff

| | |
|---|---|
| <50% of viable tumor cells with any | Negative |
| FOLR1 membrane staining visible at ≤10X | |
| microcope objective | |
| OR | |
| Absence of visible FOLR1 membrane | |
| staining at 10X microscope objective | |
| ≥50% of viable tumor cells with any | Positive |
| FOLR1 membrane staining visible at ≤10X | (Proceed to Step 2) |
| microscope objective | |

Step 2: Evaluation at the 75% cutoff

| | |
|---|---|
| <75% of viable tumor cells with any | <75% |
| FOLR1 membrane staining visible at at ≤10X | |
| microscope objective | |
| ≥75% of viable tumor cells with any | ≥75% |
| FOLR1 membrane staining visible at ≤10X | |
| microscope objective | |

Patients who are indicated as positive as described by Table 9 will receive the combination therapy as described herein.

Heterogenous/Homogenous Scoring System. A third method of cancer scoring is a heterogeneous/homogeneous scoring system. In this instance, the cancer is a cancer that expresses FOLR1 (polypeptide or nucleic acid). In some embodiments, the combination of the anti-FOLR1 immunoconjugate (e.g., IMGN853) and the anti-PD-1 antibody or antigen-binding fragment thereof (e.g., pembrolizumab) is administered to a patient with an increased expression level of FOLR1, for example, as described in U.S. Published Application No. 2012/0282175 or International Published Application No. WO 2012/135675, both of which are incorporated by reference herein in their entireties. Exemplary antibodies, assays, and kits for the detection of FOLR1 are provided in WO 2014/036495 and WO 2015/031815, both of which are incorporated by reference herein in their entireties. The FOLR1 protein expression is measured by immunohistochemistry (IHC) and given a staining intensity score and/or a staining uniformity score by comparison to controls (e.g., calibrated controls) exhibiting defined scores (e.g., an intensity score of 3 is given to the test sample if the intensity is comparable to the level 3 calibrated control or an intensity of 2 (moderate) is given to the test sample if the intensity is comparable to the level 2 calibrated control). A staining uniformity that is “heterogeneous” or “hetero” (i.e., at least 25% and less than 75% cells stained). A staining uniformity that is “homogeneous” or “homo” (i.e., at least 75% cells stained) instead of “focal” (i.e., greater than 0% and less than 25% cells stained) is also indicative of increased FOLR1 expression. The staining intensity and staining uniformity scores can be used alone or in combination (e.g., 2 homo, 2 hetero, 3 homo, 3 hetero, etc.). In another example, an increase in FOLR1 expression can be determined by detection of an increase of at least 2-fold, at least 3-fold, or at least 5-fold) relative to control values (e.g., expression level in a tissue or cell from a subject twithout cancer or with a cancer that does not have elevated FOLR1 values). The staining uniformity score can be based on the percent of stained cells.

The cancer can be a cancer that expresses FOLR1 at a level of 1 hetero or higher by IHC. The cancer can be a cancer that expresses FOLR1 at a level of 2 hetero or higher by IHC. The cancer can be a cancer that expresses FOLR1 at a level of 3 hetero or higher by IHC. The cancer can be an ovarian cancer that expresses FOLR1 at a level of 2 hetero or higher by IHC. The cancer can be an ovarian cancer that expresses FOLR1 at a level of 3 hetero or higher by IHC. The cancer also can be an endometrial cancer that expresses FOLR1 at a level of 2 hetero or higher by IHC.

A least one cell is present in a sample obtained from a patient has an FOLR1 score of at least 1. At least one cell in sample obtained from a patient can have an FOLR1 score of at least 2 (moderate). At least one cell in sample obtained from a patient can have an FOLR1 score of at least 3.

In the hetero/homo scoring system, at least 25% of the cells in a sample obtained from a patient have a FOLR1 IHC score of at least 1. In some embodiments, at least 33% of the cells in a sample obtained from a patient have a FOLR1 IHC score of at least 1. In some embodiments, at least 50% of the cells in a sample obtained from a patient have a FOLR1 IHC score of at least 1. In some embodiments, at least 66% of the cells in a sample obtained from a patient have a FOLR1 IHC score of at least 1. In some embodiments, at least 75% of the cells in a sample obtained from a patient have a FOLR1 IHC score of at least 1.

When using the hetero/homo scoring systemat least 25% of the cells in a sample obtained from a patient have a FOLR1 IHC score of at least 2 (“moderate”). In some embodiments, at least 33% of the cells in a sample obtained from a patient have a FOLR1 IHC score of at least 2 (“moderate”). In some embodiments, 25-75% of the cells in a sample obtained from a patient have a FOLR1 IHC score of at least 2 (“moderate”). In some embodiments, at least 50% of the cells in a sample obtained from a patient have a FOLR1 IHC score of at least 2 (“moderate”). In some embodiments, at least 66% of the cells in a sample obtained from a patient have a FOLR1 IHC score of at least 2 (“moderate”). In some embodiments, at least 75% of the cells in a sample obtained from a patient have a FOLR1 IHC score of at least 2 (moderate).

When using the hetero/homo scoring system, at least 25% of the cells in a sample obtained from a patient have a FOLR1 IHC score of at least 3. In some embodiments, at least 33% of the cells in a sample obtained from a patient have a FOLR1 IHC score of at least 3. In some embodiments, at least 50% of the cells in a sample obtained from a patient have a FOLR1 IHC score of at least 3. In some embodiments, at least 66% of the cells in a sample obtained from a patient have a FOLR1 IHC score of at least 3. In some embodiments, at least 75% of the cells in a sample obtained from a patient have a FOLR1 IHC score of at least 3.

### V.C. Dosing

As provided herein, an anti-FOLR1 immunoconjugate (e.g., IMGN853) can be administered at a particular dose and/or at particular timing intervals. Administration of anti-FOLR1 immunoconjugates (e.g., IMGN853) can be, for example, intravenous or intraperitoneal. Dosing regiments for anti-FORL1 immunoconjugates (e.g., IMGN853) are provided, for example, in WO 2014/186403, WO 2015/054400, and WO 2015/149018, each of which is herein incorporated by reference in its entirety.

For example, an anti-FOLR1 immunoconjugate (e.g., IMGN853) can be administered at a dose of about 6 mg/kg, wherein the kilograms of body weight are based on adjusted ideal body weight (AIBW).

In some embodiments, an anti-FOLR1 immunoconjugate (e.g., IMGN853) is administered every three weeks.

In some embodiments, an anti-FOLR1 immunoconjugate (e.g., IMGN853) is administered every three weeks at a dose of about 6 mg/kg AIBW.

An anti-FOLR1 immunoconjugate (e.g., IMGN853) can be administered at a dose of about 5 mg/kg AIBW. In some embodiments, an anti-FOLR1 immunoconjugate (e.g., IMGN853) is administered every three weeks at a dose of about 5 mg/kg AIBW.

As provided herein, an anti-PD-1 antibody or antigen-binding fragment thereof can be administered at a particular dose and/or at particular timing intervals. Administration of an anti-PD-1 antibody or antigen-binding fragment thereof (e.g., pembrolizumab) can be, for example, intravenous.

In some embodiments, an anti-PD-1 antibody or antigen-binding fragment thereof (e.g., pembrolizumab) is administered every three weeks (Q3W). In some embodiments, an anti-PD-1 antibody or antigen-binding fragment thereof (e.g., pembrolizumab) is administered at a dose of about 200 mg. In some embodiments, an anti-PD-1 antibody or antigen-binding fragment thereof (e.g., pembrolizumab) is administered every three weeks at a dose of about 200 mg.

In some embodiments, an anti-PD-1 antibody or antigen-binding fragment thereof (e.g., pembrolizumab) is administered every three weeks administered at a dose of about 200 mg and an anti-FOLR1 immunoconjugate (e.g., IMGN853) is administered every three weeks at a dose of about 6 mg/kg AIBW.

In some embodiments, an anti-PD-1 antibody or antigen-binding fragment thereof (e.g., pembrolizumab) is administered every three weeks administered at a dose of about 200 mg and an anti-FOLR1 immunoconjugate (e.g., IMGN853) is administered every three weeks at a dose of about 5 mg/kg AIBW.

In one instance, the immunoconjugate that binds to FOLR1 (e.g., IMGN853) and the anti-PD-1 antibody or antigen-binding fragment thereof (e.g., pembrolizumab) are administered simultaneously. In one instance, the anti-FOLR1 immunoconjugate (e.g., IMGN853) and the anti-PD-1 antibody or antigen-binding fragment thereof (e.g., pembrolizumab) are administered in separate pharmaceutical compositions. In one instance, the anti-FOLR1 immunoconjugate (e.g., IMGN853) and the anti-PD-1 antibody or antigen-binding fragment thereof (e.g., pembrolizumab) are administered in the same pharmaceutical composition. In one instance, the anti-FOLR1 immunoconjugate (e.g., IMGN853) and the anti-PD-1 antibody or antigen-binding fragment thereof (e.g., pembrolizumab) are administered sequentially. In one instance, the anti-FOLR1 immunoconjugate (e.g., IMGN853) and the anti-PD-1 antibody or antigen-binding fragment thereof (e.g., pembrolizumab) are administered sequentially, and the immunoconjugate is administered before the anti-PD-1 antibody or antigen-binding fragment thereof.

### V.D. Assessment and Monitoring

In certain embodiments, the combination of the anti-FOLR1 immunoconjugate (e.g., IMGN853) and the anti-PD-1 antibody or antigen-binding fragment thereof (e.g., pembrolizumab) is useful for inhibiting tumor growth. In certain embodiments, the combination of the anti-FOLR1 immunoconjugate (e.g., IMGN853) and the anti-PD-1 antibody or antigen-binding fragment thereof (e.g., pembrolizumab) is useful for reducing or preventing metastasis. In certain embodiments, the combination of the anti-FOLR1 immunoconjugate (e.g., IMGN853) and the anti-PD-1 antibody or antigen-binding fragment thereof (e.g., pembrolizumab) is useful for reducing tumor volume.

For example, in some embodiments, treatment with a combination of a FOLR1 immunoconjugate (e.g., IMGN853) and an anti-PD-1 antibody or antigen-binding fragment thereof (e.g., pembrolizumab) results in a reduction in tumor size, mass, or volume.

In some embodiments, the combination of a FOLR1 immunoconjugate (e.g., IMGN853) and an anti-PD-1 antibody or antigen-binding fragment thereof (e.g., pembrolizumab) is capable of inhibiting metastases. In certain embodiments, the combination of a FOLR1 immunoconjugate (e.g., IMGN853) and an anti-PD-1 antibody or antigen-binding fragment thereof (e.g., pembrolizumab) can reduce the tumorigenicity of a tumor. The methods of use can be in vivo methods.

In certain embodiments, the combination of a FOLR1 immunoconjugate (e.g., IMGN853) and an anti-PD-1 antibody or antigen-binding fragment thereof (e.g., pembrolizumab) produces a synergistic effect.

In certain embodiments, administration of the combination of a FOLR1 immunoconjugate (e.g., IMGN853) and an anti-PD-1 antibody or antigen-binding fragment thereof (e.g., pembrolizumab) does not produce more toxicity than administration of the anti-PD-1 antibody or antigen-binding fragment thereof. In some embodiments, administration of the combination of a FOLR1 immunoconjugate (e.g., IMGN853) and an anti-PD-1 antibody or antigen-binding fragment thereof (e.g., pembrolizumab) does not produce more toxicity than administration of the anti-FOLR1 immunoconjugate. In some embodiments, administration of the combination of a FOLR1 immunoconjugate (e.g., IMGN853) and an anti-PD-1 antibody or antigen-binding fragment thereof (e.g., pembrolizumab) does not produce more toxicity than administration of either the anti-FOLR1 immunoconjugate or the anti-PD-1 antibody or antigen-binding fragment thereof (e.g., pembrolizumab).

Each of the above aspects can further include monitoring the subject for recurrence of the cancer. Monitoring can be accomplished, for example, by evaluating progression free survival (PFS), overall survival (OS), objective response rate (ORR) complete response (CR), partial response (PR).

In one embodiment, the PFS is evaluated after initiation of treatment. In some embodiments, PFS is extended by about 3-6 months, compared to a control. In one embodiment, the PFS is extended by about 3 months with the treatment regimen combining a FOLR1 immunoconjugate (e.g., IMGN853) and an anti-PD-1 antibody or antigen-binding fragment thereof (e.g., pembrolizumab) compared to a control. In one embodiment, the PFS is extended by at least about 4 months with the treatment regimen combining a FOLR1 immunoconjugate (e.g., IMGN853) and an anti-PD-1 antibody or antigen-binding fragment thereof (e.g., pembrolizumab) compared to a control. In another embodiment, the PFS is extended by about 5 months with the treatment regimen combining a FOLR1 immunoconjugate (e.g., IMGN853) and an anti-PD-1 antibody or antigen-binding fragment thereof (e.g., pembrolizumab) compared to a control. In one embodiment, the PFS is extended by about 6 months with the treatment regimen combining a FOLR1 immunoconjugate (e.g., IMGN853) and an anti-PD-1 antibody or antigen-binding fragment thereof (e.g., pembrolizumab) compared to a control.

In one embodiment, the total PFS time after treatment with the combination of a FOLR1 immunoconjugate (e.g., IMGN853) and an anti-PD-1 antibody or antigen-binding fragment thereof (e.g., pembrolizumab) is about 3 months to 1 year. In one embodiment, the total PFS time is about 3 months. In one embodiment, the total PFS time is about 4 months. In one embodiment, the total PFS time is about 5 months. In one embodiment, the total PFS time is about 6 months. In one embodiment, the total PFS time is about 7 months. In one embodiment, the total PFS time is about 8 months. In one embodiment, the total PFS time is about 9 months. In one embodiment, the total PFS time is about 10 months. In one embodiment, the total PFS time is about 11 months. In one embodiment, the total PFS time is about 1 year. In one embodiment, the total PFS time is about 6 to 9 months. In one embodiment, the total PFS time is about 6 to 8 months.

The objective response rate (ORR) is the proportion of patients achieving a complete response partial response or stable disease (CR, PR or SD). In one embodiment, the treatment provided herein achieves an ORR of at least about 25%. In one embodiment, the treatment provided herein achieves an ORR ofabout 30%. In one embodiment, the treatment provided herein achieves an ORR of about 35%. In one embodiment, the treatment provided herein achieves an ORR of about 40%. In one embodiment, the treatment provided herein achieves an ORR of about 45%. In one embodiment, the treatment provided herein achieves an ORR ofabout 50%. In one embodiment, the treatment provided herein achieves an ORR of 25-50%.

In one embodiment, treatment with FOLR1 immunoconjugate (e.g., IMGN853) and an anti-PD-1 antibody or antigen-binding fragment thereof (e.g., pembrolizumab) increase PFS and ORR. The total PFS can be about 3 months to about 1 year and the ORR can be at least 25%. The total PFS can be about 3 months to about 1 year and the ORR can be about 25-50%. The total PFS can be about 9 months and the ORR can be at least 25%. The total PFS can be about 9 months and the ORR can be about 25-50%.

### V.E. Additional Therapies

A steroid can be administered in addition to the combination of a FOLR1 immunoconjugate (e.g., IMGN853) and an anti-PD-1 antibody or antigen-binding fragment thereof (e.g., pembrolizumab). In some embodiments, the administration of the steroid in addition to the combination of a FOLR1 immunoconjugate (e.g., IMGN853) and an anti-PD-1 antibody or antigen-binding fragment thereofresults in a reduction of headaches as compared to administration of only the combination of a FOLR1 immunoconjugate (e.g., IMGN853) and an anti-PD-1 antibody or antigen-binding fragment thereof (e.g., pembrolizumab).

The steroid can be administered at the same time as the immunoconjugate, prior to the administration of the immunoconjugate, and/or after the administration of the immunoconjugate. In some embodiments, the steroid is administered within about a week, about five days, about three days, about two days, or about one day or 24 hours prior to the administration of the immunoconjugate. In some embodiments, the steroid is administered within one day of the administration of the immunoconjugate. In some embodiments, the steroid is administered about 30 minutes prior to the administration of the immunoconjugate. In some embodiments, the steroid is administered multiple times. In some embodiments, the steroid is administered about one day prior to the administration of the immunoconjugate and on the same day as the administration of the immunoconjugate. The steroid can be administered via any number of ways, including for example, topical, pulmonary, oral, parenteral, or intracranial administration. In some embodiments, the administration is oral. In some embodiments, the administration is intravenous. In some embodiments, the administration is both oral and intravenous.

By way of example, acetaminophen/paracetamol, dexamethasone, and/or diphenhydramine can be administered prior to (e.g., 30 minutes prior to) administration of the immunoconjugate (e.g., IMGN853). For instance, 325-650 mg acetaminophen/paracetamol (orally or intravenously), 10 mg dexamethasone (intravenously), and/or 25-50 mg diphenhydramine (orally or intravenously) can be administered prior to (e.g., 30 minutes prior to) administration of the immunoconjugate (e.g., IMGN853).

In some embodiments, a steroid is administered in an eye drop (e.g., a corticosteroid eye drop, including, but not limited to: cortisol, glucocorticoid, dexamethasone, cortisone, prednisolone, fluocinolone, difluprednate, loteprednol, fluorometholone, triamcinolone, rimexolone). In some embodiments, the eye drops are preservative-free, lubricating eye drops. In some embodiments, the steroid in the eye drop is dexamethasone.

In certain embodiments, a lubricating eye drop can be administered in addition to the ophthalmic steroid, which is administered to reduce the ocular toxicity associated with the administration of an anti-FOLR1 immunoconjugate (e.g., IMGN853). The lubricating eye drop can be administered to reduce dry eye. In some embodiments, the lubricating eye drops are preservative-free, lubricating eye drops. In some embodiments, the lubricating eye drops are not administered on the same day as the ophthalmic steroid (e.g., administered after an ophthalmic steroid). In other embodiments, the lubricating eye drops are administered on the same day as the ophthalmic steroid.

Another analgesic or other medication to prevent or treat headaches can also be administered in addition to the combination of a FOLR1 immunoconjugate (e.g., IMGN853) and an anti-PD-1 antibody or antigen-binding fragment thereof (e.g., pembrolizumab). For example, acetaminophin and/or dephenhydramine can be administered in addition to the combination of a FOLR1 immunoconjugate (e.g., IMGN853) and an anti-PD-1 antibody or antigen-binding fragment thereof (e.g., pembrolizumab). The analgesic can be administered prior to, at the same time, or after the administration of the immunoconjugate and can be via any appropriate administration route. In some embodiments, the analgesic is administered orally.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### EXAMPLES

It is understood that the examples and embodiments described herein are for illustrative purposes only and that various modifications or changes in light thereof will be suggested to persons skilled in the art and are to be included within the spirit and purview of this application.

### Example 1

The safety and tolerability of the combination of IMGN853 (mirvetuximab soravtansine) administered with pembrolizumab (Keytruda®) was evaluated in a Phase 1b trial in patients with FOLR1-positive cancers that were (i) platinum resistant epithelial ovarian cancer (EOC), (ii) primary peritoneal cancer, or (iii) fallopian tube cancer. Cancers were considered FOLR1-positive where at least 25% of tumor cells had a staining intensity of 2 or greater as measured by immunohistochemistry (IHC). All patients had at least one lesion that met the definition of measurable disease according to RECIST 1.1.

The demographics and baseline characteristics of treated patients are shown in Tables 10 and 11 below.

TABLE 10
Patient Demographics and Baseline Characteristics

| | IMGN853 + Pembrolizumab | |
|---|---|---|
| Characteristic | (n = 13) | |
| Age | | |
| Median | 62 | |
| Range | 47-78 | |
| Race, n (%) | | |
| White | 13 | (100) |
| Not reported | — | |
| Primary cancer diagnosis, n (%) | | |
| Ovarian cancer | 8 | (62) |
| Fallopian tube cancer | 3 | (23) |
| Peritoneal cancer | 1 | (8) |
| Other | 1 | (8) |
| ECOG PS, n (%) | | |
| 0 | 8 | (62) |
| 1 | 5 | (38) |
| No. of prior systemic therapies, n (%) | | |
| 1-2 | 1 | (8) |
| 3 | 4 | (31) |
| 4-6 | 7 | (54) |
| 7+ | 1 | (8) |
| Median range | 5 | (2-7) |
| FOLR1 expression | | |
| High | 5 | (38) |
| Medium | 2 | (15) |
| Low | 6 | (46) |
| Prior exposure, n (%) | | |
| Platinum compounds | 13 | (100) |
| Taxanes | 13 | (100) |
| Bevacizumab | 6 | (46) |
| PARP inhibitor | 4 | (31) |

TABLE 11
Patient Demographics & Baseline Characteristics.

| Parameter | IMGN853 + pembrolizumab |
|---|---|
| Number enrolled | 13 |
| Median number of prior therapies (range) | 5 (2-7) |
| Grade 3 or greater adverse events | None |
| in > 1 patient | |
| Dose limiting toxicity | None |
| Objective response rate | NA |
| Median progression free survival (months) | NA |

Patients were treated with 6.0 mg/kg adjusted ideal body weight (AIBW) of IMGN853 followed by 200 mg pembrolizumab every three weeks (QW3) until disease progression, an adverse event, or decision to end treatment was made by the patient or an investigator.

The starting dose level of IMGN853 was 5 mg/kg AIBW. IMGN853 was administered first, followed by pembrolizumab. If that was well-tolerated, a dose of 6 mg/kg AIBW of IMGN853 was administered.

IMGN853 was administered as an intravenous (IV) infusion. Patients who had not previously been treated with IMGN853 was a prior cancer therapy received IMGN853 as a rate of 1 mg/min for 30 minutes. If that was well-tolerated, the rate was increased to 3 mg/min. If that was well-tolerated, the rate was increased to 5 mg/min, and subsequent infusions were administered at the tolerated rate.

Pembrolizumab was administerd at a dose of 200 mg using a 30-minute IV infusion.

All patients received 325-650 mg of acetaminophen/paracetamol orally (PO) or IV, 10 mg IV dexamethasone, and 25-50 mg diphenhydramine PO or IV (or equivalent drugs of similar drug classes) approximately 30 minutes prior to each infusion of IMGN853.

Treatment emergent adverse events (TEAE) that occurred with a frequency of at least 20% included diarrhea, nausea, blurred vision, fatigue, and proteinuria. The results of adverse events are listed in Tables 12 and 13.

TABLE 12
Treatment Emergent AEs: >20% (All Grades).

| | IMGN853 + Pembrolizumab | |
|---|---|---|
| Treatment emergent AE | (n = 13) | |
| Abdominal pain (%) | 1 | (7.7) |
| Constipation (%) | 2 | (15.4) |
| Decreased appetite (%) | 2 | (15.4) |
| Diarrhea (%) | 2 | (15.4) |
| Dry eye (%) | 1 | (7.7) |
| Fatigue (%) | 4 | (30.8) |
| Headache (%) | 1 | (7.7) |
| Hypertension (%) | 1 | (7.7) |
| Nausea (%) | 3 | (23.1) |
| Peripheral neuropathy*(%) | 4 | (30.8) |
| Small intestinal obstruction (%) | 1 | (7.7) |
| Vision blurred (%) | 2 | (15.4) |
| Vomiting (%) | 1 | (7.7) |

*Includes Neuropathy Peripheral, Peripheral Sensory Neuropathy, Peripheral Motor Neuropathy, Paraesthesia, and Hypoesthesia

TABLE 13
Treatment Emergent AEs: Grade 3+.

| | IMGN853 + Pembrolizumab |
|---|---|
| Treatment emergent AE | (n = 13) |
| Febrile neutropenia (%) | 1 (7.7) |
| Small intestinal obstruction (%) | 1 (7.1) |

The ORR and PFS are observed. The combination of a high ORR and a high PFS indicate that IMGN853 at a dose of 6 mg/kg AIBW given once every three weeks and pembrolizumab at a dose of 200 mg given once every three weeks are a therapeutically effective treatment.

For example, a 49 year old with stage IIIC fallopian tube cancer received treatment with the combination of IMGN853 and pembrolizumab. The cancer was previously debulked, and the patient was treated with adjuvant cisplatin and paclitaxel. The patient was subsequently treated with doxorubicin (Doxil®)/carboplatin for six cycles followed by rucaparib/placebo. Subsequent to these treatments, the patient was diagnosed with progressive diease after all of these treatments and then treated with IMGN853 and pembrolizumab. A partial response was observed after 2 cycles of treatment, and the partial response continued after 10 cycles. The patient's CA125 response (as determined by Gynocological Cancer Intergroup (GCIG criteria)) are shown in Table 14 below.

TABLE 14
CA125 Response

| Time of Measurement | CA125 Level |
|---|---|
| Screening | 128 |
| Cycle 2 | 65 |
| Cycle 4 | 6 |
| Cycle 6 | 5 |
| Cycle 8 | 6 |
| Cycle 10 | 8 |

Furthermore, tumor size was reduced in the patient during this treatment time. Thus, the combination of IMGN853 and pembrolizumab was therapeutically effective.

### Example 2

The results of patients treated with PD-1 and PD-L1 monotherapies are shown in Table 15 below.

TABLE 15
PD-(L)1 Inhibitors Activity on Ovarian Cancer

| | Nivolumab¹ | Pembrolizumab² | Avelumab³ |
|---|---|---|---|
| N | 20 | 26 | 124 |
| Population | Platinum | Failure of prior | Recurrent/ |
| | resistant | therapy (rx) | refractory |
| ORR | 15% | 11/5% | 9.7% |
| Duration of | median PFS | median PFS 1.9 mo; | median PFS |
| Response | 3.5 mo | DOR range (24.9+ to | 11.3 wks 6 |
| (DOR)/PFS | | 26.5+ mo) | of 12 responses |
| | | | ongoing |
| PD-L1 | No | Yes (membranous | No |
| Selection | 80% high | staining on at least 1% | |
| | 20% low | of tumor and associated | |
| | No correlation | inflammatory cells or | |
| | with ORR | positive straining in | |
| | | stroma) | |

¹Hamanishi J. et al, Journal of Clinical Oncology 33: 4015-4022 (2015).
²Varga, A. et al. Journal of Clinical Oncology 35: 5513 (2017).
³Disis, M. et al. Journal of Clinical Oncology 34: 5533 (2016).

This data shows that the combination of IMGN853 and pembrolizumabmay be more effective than PD-(L)-1 inhibitor monotherapies.

### Example 3

In this example, initial safety and activity findins from a phase 1 b escalation study of mirvetuximab soravtansine, a folate receptor alpha (FRα)-targeting antibody-drug conjugate (ADC), in combination with pembrolizumab in platinum-resistant epithelial ovarian cancer patients is provided. Combination chemotherapy with a platinum-based regimen remains a foundation of current first-line treatment for epiethelial oarain cancer (EOC). Unfortunately, a majority of these pateitns relapse and ultimately develop platinum-resistant disease. Mirevtuximab soravtansine has shown promising single-agent clinical activity and a favorable safety provide in heavily pretreated FRα-positive EOC patients (Moore et al., Cancer 123: 3080-3087, 2017; Moore et al., J. Clin. Oncol. 35: 1112-1118, 2017). The use of targeted agents as part of combination regiments sometimes has been shown to have improved outcomes from some human malignancies. In preclinical studies, mirvetuximab soravtansine has been shown to activate monoctyes and upregulate immunogenic cell death markers on ovarian tumor cells, providing a potential mechanistic rationale for combining the agent alongside the modality of an immune checkpoint blockage agent (Skaletskaya et al., J. Immuno. Ther. Cancer 4(suppl. 1): 73, 2016). The KEYNOTE-028 Phase 1 b study evaluating pembrolizumab as monotherapy in patients with PD-L1 positive ovarian cancer report an overall response rate of 11.5% and median progression free srivival of 1.9 months (Varga et al., J. Clin. Oncol. 35 (Suppl. 15): Abstract 5513, 2017). This example provides the data of mirvetuximab soravtansine in combination with pembrolizumab as part of the phase 1b FORWARD II trial (NCT02606305) in patients with platinum-resistant EOC as part of an ongoing Phase I trial (NCT01609556).

The object is to evaluate the safety and tolerability of mirvetuximab soravtansine when administered in combination with pembrolizumab in patients with EOC, primary peritoneal cancer, or fallopian tube cancer. The treatment schedule utilized is as follows: (1) Pembrolizumab+mirvetuximab soravtansine administered on Day 1 of a 3 week cycle (Q3W). (2) The first 4 patients were dosed with mirvetuximab soravtansine at 5 mg/kg (adjusted ideal body weight) and then the remaining 10 patients were treated at the phase 3 monotherapy dose of 6 mg/kg; pembrolizumab dosing remained constant at 200 mg for all the patients.

Patient eligibility was determined as follows: Platinum-resistant EOC, primary peritoneal, or fallopian tube cancer. At least one lesion that meets the definition of measurable disease according to RECIST 1.1. FRα positivity by IHC (≥25% of tumor cells with 2+ staining intensity). Patient demosgraphics is as follows.

| Characteristics | Pembrolizumab (n = 14) | |
|---|---|---|
| Age | | |
| Median | 63.5 | |
| Range | 47-78 | |
| Race | | |
| White | 14 | (100) |
| Primary cancer diagnosis | | |
| Epithelial ovarian cancer | 9 | (64) |
| Fallopian tube cancer | 3 | (21) |
| Primary peritoneal cancer | 1 | (7) |
| Papillary ovarian cancer | 1 | (7) |
| ECOG PS, n (%) | | |
| 0 | 8 | (57) |
| 1 | 6 | (43) |
| No. of prior systemic therapies, n (%) | | |
| 1-2 | 1 | (7) |
| 3 | 4 | (29) |
| 4-6 | 7 | (5) |
| 7+ | 2 | (14) |
| Median (range) | 4.5 | (2-7) |
| FRα expression* n (%) | | |
| High | 5 | (36) |
| Medium | 3 | (21) |
| Low | 6 | (43) |
| Prior exposure, n (%) | | |
| Platinum compounds | 14 | (100) |
| Taxanes | 14 | (100) |
| Bevacizumab | 6 | (43) |
| PARP inhibitor | 7 | (50) |

In one patient in the study, a 49 year old patient with platinum-resistant fallopian tube cancer showed partial response (PR) after two cycles of the combination treatment as shown by CT scan. The CA-125 levels were decreased from 128 at the time of screening to 65 at cycle 2, and a nadir of 5 at cycle 6. The combination therapy for the 49-year old patient was discontinued at cycle 14 due to progressive disease (a new lesion). Biomarker staining from the same patient showed intratumoral and tumor-associated stromal infiltration of both lymphocytes and macrophages.

The following is a listing of treatment emergent adverse events (AE's):

| | Grade 1 | | Grade 2 | | Grade 3 | | All Grades | |
|---|---|---|---|---|---|---|---|---|
| Adverse Event | No. | % | No. | % | No. | % | No. | % |
| Fatigue | 8 | 57 | 4 | 29 | 1 | 7 | 13 | 93 |
| Nausea | 6 | 43 | 3 | 21 | 2 | 14 | 11 | 79 |
| Diarrhea | 5 | 36 | 2 | 14 | 1 | 7 | 8 | 57 |
| Dry eye | 5 | 36 | 2 | 14 | 0 | 0 | 7 | 50 |
| Peripheral | 3 | 21 | 3 | 21 | 0 | 0 | 6 | 43 |
| neuropathy* | | | | | | | | |
| Constipation | 4 | 29 | 1 | 7 | 0 | 0 | 5 | 36 |
| Keratopathy** | 2 | 1 | 3 | 21 | 0 | 0 | 5 | 36 |
| Blurred vision | 1 | 7 | 4 | 29 | 0 | 0 | 5 | 36 |
| Decreased | 3 | 21 | 0 | 0 | 1 | 7 | 4 | 29 |
| appetite | | | | | | | | |
| Vomiting | 1 | 7 | 1 | 7 | 2 | 14 | 4 | 29 |
| Anemia | 1 | 7 | 2 | 14 | 0 | 0 | 3 | 21 |
| Arthralgia | 2 | 14 | 1 | 7 | 0 | 0 | 3 | 21 |
| Dyspnea | 2 | 14 | 1 | 7 | 0 | 0 | 3 | 21 |
| Hypokalemia | 3 | 21 | 0 | 0 | 0 | 0 | 3 | 21 |
| Insomnia | 3 | 21 | 0 | 0 | 0 | 0 | 3 | 21 |
| Pneumonitis | 3 | 21 | 0 | 0 | 0 | 0 | 3 | 21 |
| Small | 0 | 0 | 0 | 0 | 3 | 21 | 3 | 21 |
| intestinal | | | | | | | | |
| obstruction | | | | | | | | |

*= Includes neuropathy peripheral and peripheral sensory neuropathy.
**= Includes corneal epithelial microcysts, keratitis, keratopathy, and punctate keratitis.
The majority of the AEs reported were Grade 1 or 2 and manageable. Only one Grade 3 AE (small intestinal obstruction) was observed in more than 2 patients; no Grade 4 events were seen. One patient discontinued for a related AE (Grade 1 pneumonitis, possibly progressive). One drug-related death (colonic perforation) occurred on the study.

Confirmed objective response rate (ORR) and time to event endpoints are as follows:

| | | |
|---|---|---|
| | Total | Medium/High FRα |
| Endpoint | (n = 14) | (n = 8) |

| | | | | |
|---|---|---|---|---|
| ORR (confirmed) | 43% | (18, 71) | 63% | (25, 92) |
| PFS (months) | 5.2 | (1.6, 9.5) | 8.6 | (1.6, —) |
| Median | | | | |
| 95% Cl | | | | |
| DOR (weeks) | 30.1 | (14.9, —) | 36.1 | (14.9) |
| Median | | | | |
| 95% Cl | | | | |

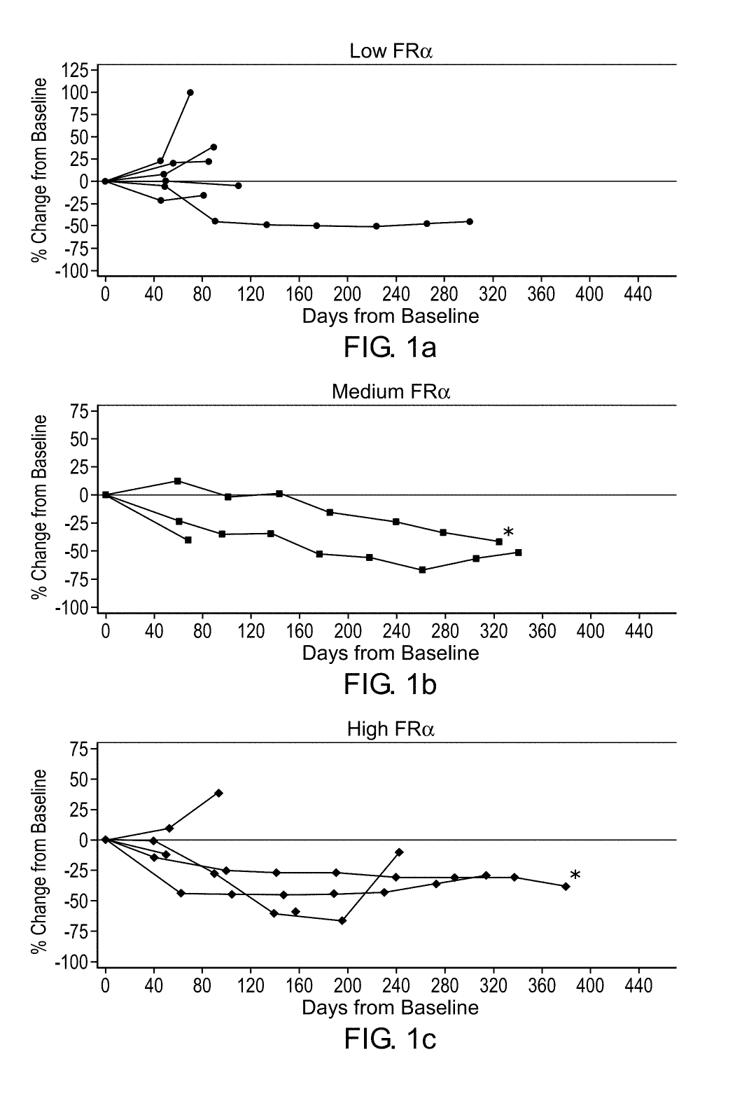

The confirmed partial responses (PRs) were observed in 6 of 14 patients treated with the mirvetuximab sorvatansine-pembrolizumab combination as part of dose-escalation. Five of these PRs occurred in individuals with medium or high FRα expression levels (i.e., ≥50% of tumor cells with 2+ staining intensity) according to the H scoring system, with two patients continuing therapy. Given these results, an expansion cohort is now being enrolled with enrichment for the FRα medium and high expressing patients, given their responsiveness over the low expressing patients. This is also shown in FIGS. 1A-C.

The conclusions at this juncture of the study is that the phase 3 monotherapy dose of mirvetuximab soravtansine was readily combined with full dose pembrolizumab, with the drug combination demonstrating favorable tolerability and encouraging signals of efficacy in patients with platinum resistant ovarian cancer. The adverse event profile was manageable and as expected based on the known event profiles of each agent. The data also showed promising early evidence of response in this heavily treated cancer population (median 4.5 prior lines of systemic therapies). In the subset of patients with medium or high FRα expression, the confirmed ORR was 63% and median PFS (progression free survival) was 8.6 months. These data support the ongoing enrollment of a total of 35 patients in an expansion cohort, with medium/high tumor FRα expression levels, to further evaluate this conmbination in the setting of platinum-resistant disease.

It is to be appreciated that the Detailed Description section, and not the Summary and Abstract sections, is intended to be used to interpret the claims. The Summary and Abstract sections sets forth one or more, but not all, exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A method for treating a patient having ovarian cancer, peritoneal cancer, or fallopian tube cancer comprising administering to said patient in need thereof:
an immunoconjugate that binds to FOLR1, wherein said immunoconjugate comprises a maytansinoid and an anti-FOLR1 antibody or antigen-binding fragment thereof comprising a heavy chain variable region (VH) complementary determining region (CDR)1 sequence of SEQ ID NO:9, a VH CDR2 sequence of SEQ ID NO:10, and a VH CDR3 sequence of SEQ ID NO:12, and a light chain variable region (VL) CDR1 sequence of SEQ ID NO:6, a VL CDR2 sequence of SEQ ID NO:7, and a VL CDR3 sequence of SEQ ID NO:8, and
an anti-PD-1 antibody or antigen-binding fragment thereof comprising a VII CDR1 sequence of SEQ ID NO:20, a VH CDR2 sequence of SEQ ID NO:21, and a VH CDR3 sequence of SEQ ID NO:22, and a VL CDR1 sequence of SEQ ID NO:23, a VL CDR2 sequence of SEQ ID NO:24, and a VL CDR3 sequence of SEQ ID NO:25.

2. The method of claim 1, wherein the anti-FOLR1 antibody or antigen-binding fragment thereof comprises a VH comprising the sequence of SEQ ID NO:3 and a VL comprising the sequence of SEQ ID NO:5.

3. The method of claim 2, wherein the anti-FOLR1 antibody or antigen-binding fragment comprises a heavy chain comprising the sequence of SEQ ID NO:13 and a light chain comprising the sequence SEQ ID NO:15.

4. The method of claim 1, wherein the maytansinoid is DM4.

5. The method of claim 1, wherein maytanisonid is linked to the antibody or antigen-binding fragement thereof by a sulfo-SPDB linker.

6. A method for treating a patient having ovarian cancer, peritoneal cancer, or fallopian tube cancer comprising administering to said patient in need thereof
an immunoconjugate that binds to FOLR1, wherein said immunoconjugate comprises a maytansinoid and an anti-FOLR1 antibody or antigen-binding fragment thereof comprising (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774; and
an anti-PD-1 antibody or antigen-binding fragment thereof comprising a VH CDR1 sequence of SEQ ID NO:20, a VH CDR2 sequence of SEQ ID NO:21, and a VH CDR3 sequence of SEQ ID NO:22, and a VL CDR1 sequence of SEQ ID NO:23, a VL CDR2 sequence of SEQ ID NO:24, and a VL CDR3 sequence of SEQ ID NO:25.

7. The method of claim 6, wherein the the maytansinoid is DM4, and wherein the DM4 is linked to the antibody by sulfo-SPDB.

8. The method of claim 1, wherein the immunoconjugate comprises 1-10 maytansinoid molecules, 2-5 maytansinoid molecules, or 3-4 maytansinoid molecules.

9. The method of claim 1, wherein the immunoconjugate has the following chemical structure: 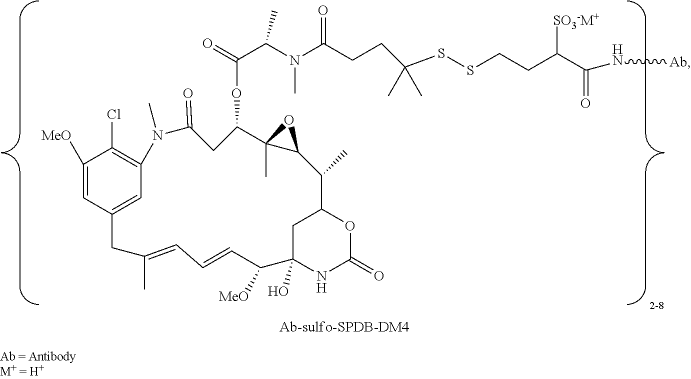
wherein “Ab” represents the anti-FOLR1 antibody or antigen binding fragment thereof.

10. The method of claim 9, wherein the immunoconjugate comprises 2-5 or 3-4 maytansinoid molecules.

11. The method of claim 1, wherein the immunoconjugate is administered once every three weeks.

12. The method of claim 1, wherein the immunoconjugate is administered at a dose of about 6 mg/kg AIBW.

13. The method of claim 1, wherein the anti-PD-1 antibody or antigen-binding fragment thereof comprises a VH comprising the sequence of SEQ ID NO:26 and a VL comprising the sequence of SEQ ID NO:27.

14. The method of claim 13, wherein the anti-PD-1 antibody or antigen-binding fragment thereof is pembrolizumab.

15. The method of claim 1, wherein the anti-PD-1 antibody or antigen-binding fragment thereof is administered once every 3 weeks.

16. The method of claim 1, wherein the anti-PD-1 antibody or antigen-binding fragment thereof is administered at a dose of about 200 mg.

17. The method of claim 1, wherein the cancer is ovarian cancer.

18. The method of claim 17, wherein the ovarian cancer is epithelial ovarian cancer.

19. The method of claim 17, wherein the ovarian cancer is platinum resistant, relapsed, or refractory.

20. The method of claim 17, wherein the administration results in a decrease in CA125.

21. The method of claim 1, wherein the peritoneal cancer is primary peritoneal cancer.

22. The method of claim 1, wherein the cancer expresses FOLR1 protein.

23. The method of claim 22, wherein the FOLR1 protein expression has been measured by immunohistochemistry (IHC) in a tumor sample obtained from the patient.

24. The method of claim 23, wherein a staining score of at least 1 hetero, at least 1 homo, at least 2 hetero, at least 2 homo, or at least 3 hetero has been measured by the IHC.

25. The method of claim 23, wherein at least 25%, at least 33%, at least 50%, at least 66%, or at least 75% of cells in the sample obtained from the patient have an IHC score of at least 2.

26. The method of claim 23, wherein at least 25%, at least 33%, at least 50%, at least 66%, or at least 75% of cells in the sample obtained from the patient have an IHC score of at least 3.

27. The method of claim 23, wherein the patient is determined to be FRα positive.

28. The method of claim 27, wherein FRα positive comprises at least 50% of tumor cells having FOLR1 membrane staining visible at less than or equal to 10× microscope objective.

29. The method of claim 23, wherein at least 25% of cells in the sample obtained from the patient have an IHC score of at least 2.

30. The method of claim 29, wherein at least 25% to no more than 49% of cells in the sample have an IHC score of at least 2.

31. The method of claim 29, wherein at least 50% to no more than 74% of cells in the sample have an IHC score of at least 2.

32. The method of claim 29, wherein at least 75% to 100% of cells in the sample have an IHC score of at least 2.

33. The method of claim 1, wherein the cancer expresses PD-L1.

34. The method of claim 1, wherein the patient has at lease one lesion that meets the definition of measurable disease according to RECIST 1.1.

35. The method of claim 1, wherein the immunoconjugate and the anti-PD-1 antibody or antigen-binding fragment thereof are administered sequentially in separate pharmaceutical compositions.

36. The method of claim 35, wherein the immunoconjugate is administered before the anti-PD-1 antibody or antigen-binding fragment thereof.

37. The method of claim 1, wherein the immunoconjugate is administered intravenously or intraperitoneally.

38. The method of claim 1, wherein the anti-PD-1 antibody or antigen-binding fragment thereof is administered intravenously.

39. The method of claim 1, wherein administration is a first-line therapy.

40. The method of claim 1, wherein administration is a second-line therapy.

41. The method of claim 1, wherein the administration is a third-line or later than third line therapy.

42. The method of claim 1, wherein the patient has previously been treated with a platinum compound, a taxane, bevacizumab, a PARP inhibitor, or a combination thereof.

43. The method of claim 1, wherein the cancer is primary platinum refractory.

44. The method of claim 1, wherein the cancer is platinum resistant.

45. The method of claim 1, wherein the cancer is platinum sensitive.

46. The method of claim 1, wherein the cancer is metastatic or advanced.

47. The method of claim 1, wherein administration of the immunoconjugate with the anti-PD-1 antibody or antigen-binding fragment thereof produces a greater therapeutic benefit than administration of the immunoconjugate alone or the anti-PD-1 antibody or antigen-binding fragment thereof alone.

48. The method of claim 1, wherein administration of the immunoconjugate and the anti-PD-1 antibody or antigen-binding fragment thereof does not produce more toxicity than administration of the immunoconjugate alone or the anti-PD-1 antibody or antigen-binding fragment thereof alone.

49. The method of claim 1, further comprising administering a steroid to the patient.

50. The method of claim 49, wherein the steroid is administered prior to the administration of the immunoconjugate.

51. The method of claim 50, wherein the steroid is administered about 30 minutes prior to the administration of the immunoconjugate.

52. The method of claim 49, wherein the steroid is a corticosteroid.

53. The method of claim 49, wherein the steroid is dexamethasone.

54. The method of claim 49, wherein the steroid is administered orally, intravenously, or a combination thereof.

55. The method of claim 49, wherein the steroid is administered as an eye drop.

56. The method of claim 49, wherein the eye drop is a lubricating eyedrop.

57. The method of claim 1, further comprising administering acetaminophen, diphenhydramine, or a combination thereof to the patient.

58. A method of treating a patient having ovarian, peritoneal, or fallopian tube cancer comprising administering to said patient in need thereof 6 mg/AIBW kg of an immunoconjugate that binds to FOLR1 and 200 mg of pembrolizumab,
wherein the immunoconjugate that binds to FOLR1 comprises an antibody linked to the maytansinoid DM4 by a sulfo-SPDB linker, wherein the an antibody comprises (i) a heavy chain comprising the sequence of SEQ ID NO:13 and (ii) a light chain comprising the sequence of SEQ ID NO:15.

59. A method of treating a patient having ovarian, peritoneal, or fallopian tube cancer comprising administering to said patient in need thereof 6 mg/AIBW kg of an immunoconjugate that binds to FOLR1 and 200 mg of pembrolizumab,
wherein the immunoconjugate that binds to FOLR1 comprises an antibody linked to the maytansinoid DM4 by a sulfo-SPDB linker, wherein the an antibody comprises (i) a heavy chain comprising the same amino acid sequence as the amino acid sequence of the heavy chain encoded by the plasmid deposited with the American Type Culture Collection (ATCC) as PTA-10772 and (ii) a light chain comprising the same amino acid sequence as the amino acid sequence of the light chain encoded by the plasmid deposited with the ATCC as PTA-10774.

60. The method of claim 58, wherein the immunoconjugate comprises 1-10, 2-5, or 3-4 maytansinoids.

61. The method of claim 58, wherein the immunoconjugate has the following chemical structure: 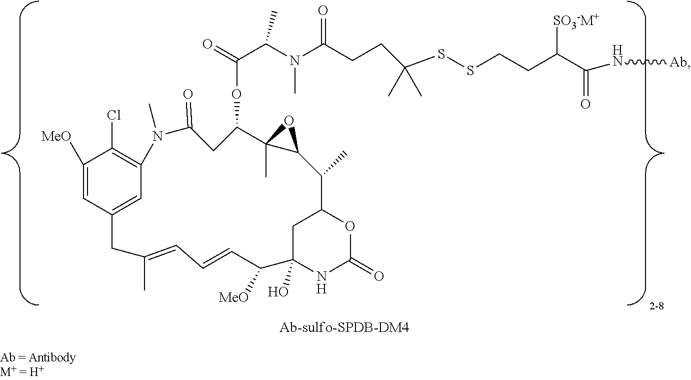
wherein “Ab” represents the anti-FOLR1 antibody or antigen binding fragment thereof.

62. The method of claim 61, wherein the immunoconjugate comprises 2-5 or 3-4 maytansinoids.

63. The method of claim 58, wherein at least 25% of cells in a tumor sample obtained from the patient have an FOLR1 IHC score of at least 2.

64. The method of claim 58, wherein the immunoconjugate and the pembrolizumab are administered intravenously, and the immunoconjugate is administered before the pembrolizumab.

65. The method of claim 58, wherein a steroid is administered prior to the administration of the immunoconjugate.

66. The method of claim 1, wherein said patient has medium or high FRα expression on the ovarian cancer, the peritoneal cancer or the fallopian tube cancer.

67. The method of claim 1, wherein said patient has FOLR1 positive status.
